(19) United States
(12) Patent Application Publication
(10) Pub. No.: US 2023/0348373 A1
(43) Pub. Date: Nov. 2, 2023

(54) DEUTERATED DERIVATIVES OF 6,8-BIS(BENZYLSULFANYL)OCTANOIC ACID

(71) Applicant: Rafael Pharmaceuticals, Inc., Cranbury, NJ (US)

(72) Inventors: Sol J. Barer, Mendham, NJ (US); Yanchao Xin, Levittown, PA (US); Yaping Hong, Levittown, PA (US); Gang Zhou, Bridgewater, NJ (US)

(73) Assignee: Rafael Pharmaceuticals, Inc., Cranbury, NJ (US)

(21) Appl. No.: 17/626,061

(22) PCT Filed: Jul. 10, 2020
(86) PCT No.: PCT/US2020/041503

§ 371 Date: Jan. 10, 2022

Related U.S. Application Data
(60) Provisional application No. 63/028,166, filed on May 21, 2020.
(60) Provisional application No. 62/873,447, filed on Jul. 12, 2019.

(51) Int. Cl. C07C 323/52; C07C 323/41; C07D 295/16; A61P 35/00

(52) U.S. Cl. CPC C07C 323/52; A61P 35/00; C07C 323/41; C07D 295/16; C07B 2200/05

(57) ABSTRACT

The invention provides new deuterated derivatives of 6,8-bis(benzylsulfanyl)octanoic acid, pharmaceutical compositions thereof, and methods for treating cancer with the new compositions.

### CROSS-REFERENCE TO RELATED APPLICATIONS

This application is the national stage application of International (PCT) Pat. Application Serial No. PCT/US2020/041503, filed Jul. 10, 2020, which claims the benefit of and priority to U.S Provisional Pat. Application Serial No. 62/873,447, filed Jul. 12, 2019 and U.S Provisional Pat. Application Serial No. 63/028,166 filed May 21, 2020; the contents of each of which are hereby incorporated by reference.

### FIELD OF THE INVENTION

The invention provides new compounds for treating cancer.

### BACKGROUND

CPI-613 (6,8-bis(benzylsulfanyl)octanoic acid) is a first-in-class investigational small-molecule (lipoate analog), which targets the altered energy metabolism unique to many cancer cells. CPI-613 is currently being evaluated in two phase III clinical trials, and has been granted orphan drug designation for the treatment of pancreatic cancer, acute myeloid leukemia (AML), peripheral T-cell lymphoma (PTCL), Burkitt lymphoma and myelodysplastic syndromes (MDS).

One limitation to the clinical utility of CPI-613 is its very rapid metabolism. After IV dosing the half-life of 6,8-bis(benzylsulfanyl)octanoic acid is only about 1-2 hours (Pardee, T.S. et al., Clin Cancer Res.Cancer Res. 2014, 20, 5255-64). The short half-life limits the patient’s overall exposure to the drug and necessitates administration of relatively high doses. For safety reasons, CPI-613 is administered via a central venous catheter as an IV infusion over 30-120 minutes, with higher doses requiring longer infusion times.

A need exists for new derivatives of CPI-613 with improved pharmacokinetic properties. The present invention addresses this need and provides other related advantages.

### SUMMARY

The invention provides new deuterated derivatives of 6,8-bis(benzylsulfanyl)octanoic acid and salts thereof, and compositions containing the new compounds. The invention also provides methods for treating cancer using the new compounds and compositions. The cancer may be a lymphoma, leukemia, carcinoma, sarcoma, melanoma, myeloma, brain or spinal cord cancer, blastoma, germ cell tumor, cancer of the pancreas, colorectal cancer, myelodysplastic syndrome, or cancer of the prostate. In certain embodiments, the cancer is a lymphoma, leukemia, carcinoma, sarcoma, melanoma, or myeloma.

The foregoing aspects of the invention are described in more detail, along with additional embodiments, in the detailed description below.

### DETAILED DESCRIPTION

### I. Definitions

To facilitate an understanding of the present invention, a number of terms and phrases are defined below.

The terms “6,8-bis(benzylsulfanyl)octanoic acid” and “6,8-bis-benzylthio-octanoic acid” refer to the compound known as CPI-613 or devimistat, having the chemical structure

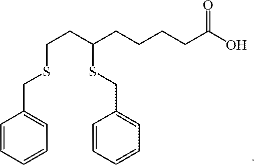

Certain compounds contained in compositions of the present invention may exist in particular geometric or stereoisomeric forms. The present invention contemplates all such compounds, including cis- and trans-isomers, R- and S-enantiomers, diastereomers, (_{D})-isomers, (_{L})-isomers, the racemic mixtures thereof, and other mixtures thereof, as falling within the scope of the invention.

“Alkyl” or “alkyl group” refers to a monoradical of a branched or unbranched saturated hydrocarbon chain. Examples include, but are not limited to, methyl, ethyl, n-propyl, n-butyl, n-pentyl, n-hexyl, n-heptyl, n-octyl, n-nonyl, n-decyl, isopropyl, tert-butyl, isobutyl, etc. Alkyl groups typically contain 1-10 carbon atoms, such as 1-6 carbon atoms or 1-4 carbon atoms, and can be substituted or unsubstituted.

“Halogen” refers to fluorine, chlorine, bromine and iodine atoms.

“Heterocycloalkyl” or “heterocycloalkyl group” refers to 3-15 membered monocyclic, bicyclic, and tricyclic non-aromatic rings, which may be saturated or unsaturated, can be substituted or unsubstituted, may be bridged, spiro, and/or fused, and which contain, in addition to carbon atom(s), at least one heteroatom, such as nitrogen, oxygen, sulfur or phosphorus. Examples include, but are not limited to, tetrahydrofuranyl, pyrrolidinyl, pyrrolinyl, imidazolidinyl, imidazolinyl, pyrazolidinyl, pyrazolinyl, piperidyl, piperazinyl, indolinyl, isoindolinyl, morpholinyl, thiomorpholinyl, homomorpholinyl, homopiperidyl, homopiperazinyl, thiomorpholinyl-5-oxide, thiomorpholinyl-S,S-dioxide, pyrrolidinyl, tetrahydropyranyl, piperidinyl, tetrahydrothienyl, homopiperidinyl, homothiomorpholinyl-S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl-5-oxide, tetrahydrothienyl-S,S-dioxide, homothiomorpholinyl-5-oxide, quinuclidinyl, 2-oxa-5-azabicyclo[2.2.1]heptanyl, 8-oxa-3-aza-bicyclo[3.2.1]octanyl, 3,8-diaza-bicyclo[3.2.1]octanyl, 2,5-diaza-bicyclo[2.2.1]heptanyl, 3,8-diaza-bicyclo[3.2.1]octanyl, 3,9-diaza-bicyclo[4.2.1]nonanyl, 2,6-diaza-bicyclo[3.2.2]nonanyl, [1,4]oxaphosphinanyl- 4-oxide, [1,4]azaphosphinanyl- 4-oxide, [1,2]oxaphospholanyl- 2-oxide, phosphinanyl-1-oxide, [1,3]azaphospholidinyl- 3-oxide, [1,3]oxaphospholanyl- 3-oxide and 7-oxabicyclo[2.2.1]heptanyl. A heterocycloalkyl group may contain, in addition to carbon atom(s), at least one nitrogen, oxygen, or sulfur. For example, a heterocycloalkyl group may contain, in addition to carbon atom(s), at least one nitrogen or oxygen. A heterocycloalkyl group may contain, in addition to carbon atom(s), at least one nitrogen. A heterocycloalkyl group may contain carbon atoms and 1 or 2 nitrogen atoms. A heterocycloalkyl group may contain carbon atoms and an oxygen atom. A heterocycloalkyl group may contain carbon atoms, a nitrogen atom, and an oxygen atom. A heterocycloalkyl group may contain carbon atoms, a nitrogen atom, and a sulfur atom. A heterocycloalkyl group may contain carbon atoms and a sulfur atom. In certain embodiments, a heterocycloalkyl group may contain from 3 to 10 ring atoms. In certain embodiments, a heterocycloalkyl group may contain from 3 to 7 ring atoms. In certain embodiments, a heterocycloalkyl group may contain from 5 to 7 ring atoms, such as 5 ring atoms, 6 ring atoms, or 7 ring atoms. Unless otherwise indicated, the foregoing heterocycloalkyl groups can be C- attached or N-attached where such is possible and results in the creation of a stable structure. For example, piperidinyl can be piperidin-1-yl (N-attached) or piperidin-4-yl (C-attached).

As used herein, the term “patient” refers to a human being in need of medical treatment.

As used herein, the term “treating” includes any effect, e.g., lessening, reducing, modulating, ameliorating or eliminating, that results in the improvement, stabilization, or slowing progression of a condition, disease, disorder, or the like, or a symptom thereof. For example, treatment can include diminishment of a symptom of a disorder or complete eradication of a disorder. As another example, treatment can include slowing the progression of a disease, or preventing or delaying its recurrence, such as maintenance treatment to prevent or delay relapse.

“Therapeutically effective amount” refers to an amount of a compound sufficient to inhibit, halt, or cause an improvement in a disorder or condition being treated in a particular patient or patient population. For example, a therapeutically effective amount can be an amount of drug sufficient to slow the progression of a disease, or to prevent or delay its recurrence, such as maintenance treatment to prevent or delay relapse. In a human or other mammal, a therapeutically effective amount can be determined experimentally in a laboratory or clinical setting, or may be the amount required by the guidelines of the United States Food and Drug Administration, or equivalent foreign agency, for the particular disease and subject being treated. It should be appreciated that determination of proper dosage forms, dosage amounts, and routes of administration is within the level of ordinary skill in the pharmaceutical and medical arts.

As used herein, the term “pharmaceutical composition” refers to the combination of an active agent with an excipient, inert or active, suitable for administration to a human being.

The phrase “pharmaceutically acceptable” is employed herein to refer to those compounds, materials, compositions, and/or dosage forms which are, within the scope of sound judgment, suitable for use in contact with the tissues of human beings with acceptable toxicity, irritation, allergic response, and other problems or complications commensurate with a reasonable benefit/risk ratio.

As used herein, the term “pharmaceutically acceptable excipient” refers to any of the standard pharmaceutical excipients suitable for use in humans. For examples of excipients, see e.g., Martin, Remington’s Pharmaceutical Sciences, 15th Ed., Mack Publ. Co., Easton, PA [1975].

As used herein, the term “pharmaceutically acceptable salt” refers to any salt (e.g., acid or base) of a compound of the present invention which is suitable for administration to a human being. “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Examples of bases include, but are not limited to, alkali metals (e.g., sodium) hydroxides, alkaline earth metals (e.g., magnesium), hydroxides, ammonia, and compounds of formula NR₃, wherein R is CI-4 alkyl, and the like.

Further examples of salts include salts made using the ion pairing agents described in U.S. Pat. No. 8,263,653, the entire disclosure of which is incorporated by reference herein. Still further ion pairing agents can be selected with guidance from Handbook of Pharmaceutical Salts Properties, Selection and Use, IUPAC, Wiley-VCH, P.H. Stahl, ed., the entire disclosure of which is incorporated by reference herein.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited steps.

As a general matter, compositions specifying a percentage are by weight unless otherwise specified.

Unless otherwise stated, when a position is designated specifically as “H” or “hydrogen,” the position is understood to have hydrogen at its natural abundance isotopic composition which is a mixture of ¹H (protium), ²H (deuterium), and ³H (tritium).

Unless otherwise stated, when a position is designated specifically as “D” or “deuterium,” the position is understood to have deuterium at an abundance that is at least 3334 times greater than the natural abundance of deuterium, which is 0.015 percent (i.e., the position has greater than 50 percent incorporation of deuterium).

The term “isotopic enrichment factor” as used herein means the ratio between the isotopic abundance of a specified isotope at a specified position and the natural abundance of that isotope.

In preferred embodiments, a compound of this invention has an isotopic enrichment factor for each atom designated as “D” or “deuterium” of at least 3500 (52.5 percent deuterium incorporation at each designated deuterium atom), at least 4000 (60 percent deuterium incorporation), at least 4500 (67.5 percent deuterium incorporation), at least 5000 (75 percent deuterium), at least 5500 (82.5 percent deuterium incorporation), at least 6000 (90 percent deuterium incorporation), at least 6333.3 (95 percent deuterium incorporation), at least 6466.7 (97 percent deuterium incorporation), at least 6600 (99 percent deuterium incorporation), or at least 6633.3 (99.5 percent deuterium incorporation).

The term “isotopologue” refers to a compound that differs from another compound only in the isotopic composition thereof.

The term “compound,” when referring to a compound of this invention, refers to a collection of molecules having an identical chemical structure, except that there may be isotopic variation among the constituent atoms of the molecules. In other words, a compound may - and generally will - comprise a collection of isotopologues. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. In certain embodiments, the total amount of such isotopologues relative to the most prevalent species will be less than 50 percent, less than 40 percent, less than 30 percent, less than 25 percent, less than 15 percent, less than 10 percent, less than 5 percent, less than 3 percent, less than 1 percent, or less than 0.5 percent of the compound.

### I. Therapeutic Compounds

The present invention provides a compound of formula (I):

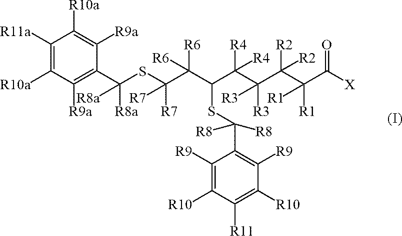

or a salt thereof, wherein
X is —OH or -NR20R21;
each R1, R3, R4, R5, R6, R7, R8, R8a, R9, R9a, R10, R10a, R11, and R11a is independently chosen from hydrogen and deuterium;
each R2 is independently chosen from hydrogen, deuterium, —OH, and —O(C₁-C₆ alkyl);
R20 and R21 are independently chosen from H and C₁-C₄ alkyl; or R20 and R21 are taken together with the nitrogen atom to which they are attached to form a 5-10 membered heterocycloalkyl, which is optionally substituted by one or more R30 groups;
each R30 is independently chosen from halogen, C₁-C₆ alkyl, —OH, —O(C₁-C₆ alkyl), —NH₂, -NH(C₁-C₆ alkyl), and -N(C_{I}-C₆ alkyl)₂;
when any combination of R1, R2, R3, R4, R5, R6, R7, R8, R8a, R9, R9a, R10, R10a, R11, and R11a is deuterium, each deuterated position has greater than 50 percent incorporation of deuterium; and
when each R1, R3, R4, R5, R6, R7, R8, R8a, R9, R9a, R10, R10a, R11, and R11a is hydrogen, then at least one R2 is deuterium, —OH, or -O(C₁-C₆ alkyl).

In certain embodiments, X is —OH. In certain embodiments, X is -NR20R21.

In certain embodiments, R20 and R21 are independently chosen from H and methyl. In certain embodiments, R20 and R21 are each independently chosen from C₁-C₄ alkyl. In certain embodiments, R20 and R21 are both methyl. In certain embodiments, R20 and R21 are both H. In certain embodiments, R20 and R21 are taken together with the nitrogen atom to which they are attached to form a 5-10 membered heterocycloalkyl, which is optionally substituted by one or more R30 groups. In certain embodiments, R20 and R21 are taken together with the nitrogen atom to which they are attached to form a 5 or 6 membered heterocycloalkyl optionally substituted by one or more R30 groups. In certain embodiments, R20 and R21 are taken together with the nitrogen atom to which they are attached to form a 6 membered heterocycloalkyl optionally substituted by one or more R30 groups. In certain embodiments, R20 and R21 are taken together with the nitrogen atom to which they are attached to form a piperazinyl group optionally substituted by one or more R30 groups. In certain embodiments, R20 and R21 are taken together with the nitrogen atom to which they are attached to form a piperazinyl group.

In certain embodiments, each R30 is independently chosen from halogen, C₁-C₄ alkyl, —OH, -O(C₁-C₄ alkyl), —NH₂, -NH(C₁-C₄ alkyl), and -N(C₁-C₄ alkyl)₂. In certain embodiments, each R30 is independently chosen from C₁-C₆ alkyl, —NH₂, and -N(C₁-C₆ alkyl)₂. In certain embodiments, each R30 is independently chosen from C₁-C₄ alkyl, —NH₂, and -N(C₁-C₄ alkyl)₂.

In certain embodiments, each R2 is independently chosen from hydrogen, deuterium, —OH, and -O(C₁-C₄ alkyl). In certain embodiments, each R2 is independently chosen from hydrogen, deuterium, and —OH. In certain embodiments, each R2 is independently chosen from deuterium and —OH. In certain embodiments, each R2 is independently chosen from hydrogen and deuterium. In certain embodiment, each R2 is independently chosen from hydrogen, —OH, and -O(C₁-C₆ alkyl). In certain embodiments, one R2 is hydrogen and the other R2 is —OH. In certain embodiments, one R2 is deuterium and the other R2 is —OH.

In certain embodiments, each R1, R3, R4, R5, R6, R7, R8, R8a, R9, R9a, R10, R10a, R11, and R11a is hydrogen, one R2 is hydrogen, and the other R2 is chosen from —OH and -O(C_{I}-C₆ alkyl). In certain embodiments, each R1, R3, R4, R5, R6, R7, R8, R8a, R9, R9a, R10, R10a, R11, and R11a is hydrogen, and each R2 is independently chosen from —OH and -O(C₁-C₆ alkyl). In certain embodiments, each R1, R3, R4, R5, R6, R7, R8, R8a, R9, R9a, R10, R10a, R11, and R11a is hydrogen and each R2 is —OH. In certain embodiments, each R1, R3, R4, R5, R6, R7, R8, R8a, R9, R9a, R10, R10a, R11, and R11a is hydrogen, one R2 is hydrogen, and the other R2 is —OH. In certain embodiments, each R1, R3, R4, R5, R6, R7, R8, R8a, R9, R9a, R10, R10a, R11, and R11a is hydrogen, one R2 is hydrogen, and the other R2 is -O(C₁-C₆ alkyl). In certain embodiments, each R1, R3, R4, R5, R6, R7, R8, R8a, R9, R9a, R10, R10a, R11, and R11a is hydrogen, one R2 is deuterium, and the other R2 is —OH.

In certain embodiments, each R1, R3, R4, R5, R6, R7, R8, R8a, R9, R9a, R10, R10a, R11, and R11a is hydrogen and each R2 is deuterium. In certain embodiments, each R1, R4, R5, R6, R7, R8, R8a, R9, R9a, R10, R10a, R11, and R11a is hydrogen and each R2 and R3 is deuterium. In certain embodiments, each R1, R4, R5, R6, R7, R8, R8a, R9, R9a, R10, R10a, R11, and R11a is hydrogen, one R2 and one R3 is deuterium, and one R2 and one R3 is hydrogen. In certain embodiments, each R1, R5, R8, R8a, R9, R9a, R10, R10a, R11, and R11a is hydrogen and each R2, R3, R4, R6, and R7 is deuterium. In certain embodiments, each R1, R5, R6, R7, R8, R8a, R9, R9a, R10, R10a, R11, and R11a is hydrogen and each R2, R3, and R4 is deuterium. In certain embodiments, each R1, R2, R3, R4, R5, R6, R7, R8, R8a, R9, R9a, R10, R10a, R11, and R11a is deuterium. In certain embodiments, each R2, R3, R4, R5, R6, R7, R8, R8a, R9, R9a, R10, R10a, R11, and R11a is deuterium and each R1 is hydrogen. In certain embodiments, each R5, R8, R8a, R9, R9a, R10, R10a, R11, and R11a is hydrogen and each R1, R2, R3, R4, R6, and R7 is deuterium. In certain embodiments, each R8, R8a, R9, R9a, R10, R10a, R11, and R11a is hydrogen and each R1, R2, R3, R4, R5, R6, and R7 is deuterium. In certain embodiments, each R1, R8, R8a, R9, R9a, R10, R10a, R11, and R11a is hydrogen and each R2, R3, R4, R5, R6, and R7 is deuterium. In certain embodiments, each R1, R4, R5, R6, and R7 is hydrogen and each R2, R3, R8, R8a, R9, R9a, R10, R10a, R11, and R11a is deuterium. In certain embodiments, each R1, R3, R4, R5, R6, and R7 is hydrogen and each R2, R8, R8a, R9, R9a, R10, R10a, R11, and R11a is deuterium. In certain embodiments, each R1, R3, R4, R5, R6, R7, R9, R9a, R10, R10a, R11, and R11a is hydrogen and each R2, R8, and R8a is deuterium. In certain embodiments, the invention provides a salt of any of the aforementioned compounds; in certain embodiments, the salt is pharmaceutically acceptable.

In certain embodiments, the compound is a compound of formula (I-1):

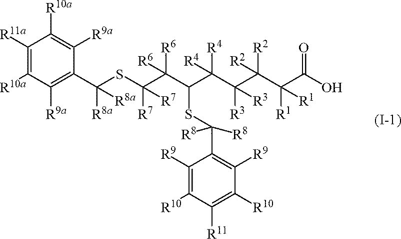

or a salt thereof, wherein R1, R², R³, R⁴, R⁵, R⁶, R⁷, R⁸, R^{8a}, R⁹, R^{9a}, R¹⁰, R^{10a}, R11, and R^{11a} are independently selected from hydrogen and deuterium; provided that when each R¹, R³, R⁴, R⁵, R⁶, R⁷, R⁸, R^{8a}, R⁹, R^{9a}, R¹⁰, R^{10a}, R11, and R^{11a} is hydrogen, then at least one R² is deuterium.

In certain embodiments, each R1, R³, R⁴, R⁵, R⁶, R⁷, R⁸, R^{8a}, R⁹, R^{9a}, R¹⁰, R^{10a}, R11, and R^{11a} is hydrogen and each R² is deuterium. In certain embodiments, each R1, R⁴, R⁵, R⁶, R⁷, R⁸, R^{8a}, R⁹, R^{9a}, R¹⁰, R^{10a}, R¹¹, and R^{11a} is hydrogen and each R² and R³ is deuterium. In certain embodiments, each R¹, R⁴, R⁵, R⁶, R⁷, R⁸, R^{8a}, R⁹, R^{9a}, R¹⁰, R^{10a}, R11, and R^{11a} is hydrogen, one R² and one R³ is deuterium, and one R² and one R³ is hydrogen. In certain embodiments, each R1, R⁵, R⁸, R^{8a}, R⁹, R^{9a}, R¹⁰, R^{10a}, R11, and R^{11a} is hydrogen and each R², R³, R⁴, R⁶, and R⁷ is deuterium. In certain embodiments, each R1, R⁵, R⁶, R⁷, R⁸, R^{8a}, R⁹, R^{9a}, R¹⁰, R^{10a}, R11, and R^{11a} is hydrogen and each R², R³, and R⁴ is deuterium. In certain embodiments, each R¹, R², R³, R⁴, R⁵, R⁶, R⁷, R⁸, R^{8a}, R⁹, R^{9a}, R¹⁰, R^{10a}, R11, and R^{11a} is deuterium. In certain embodiments, each R², R³, R⁴, R⁵, R⁶, R⁷, R⁸, R^{8a}, R⁹, R⁹ª, R¹⁰, R^{10a}, R11, and R^{11a} is deuterium and each R¹ is hydrogen. In certain embodiments, each R⁵, R⁸, R^{8a}, R⁹, R^{9a}, R¹⁰, R^{10a}, R11, and R^{11a} is hydrogen and each R¹, R², R³, R⁴, R⁶, and R⁷ is deuterium. In certain embodiments, each R⁸, R^{8a}, R⁹, R^{9a}, R¹⁰, R^{10a}, R¹¹, and R^{11a} is hydrogen and each R¹, R², R³, R⁴, R⁵, R⁶, and R⁷ is deuterium. In certain embodiments, each R¹, R⁸, R^{8a}, R⁹, R^{9a}, R¹⁰, R^{10a}, R11, and R^{11a} is hydrogen and each R², R³, R⁴, R⁵, R⁶, and R⁷ is deuterium. In certain embodiments, each R¹, R⁴, R⁵, R⁶, and R⁷ is hydrogen and each R², R³, R⁸, R^{8a}, R⁹, R^{9a}, R¹⁰, R^{10a}, R11, and R^{11a} is deuterium. In certain embodiments, each R¹, R³, R⁴, R⁵, R⁶, and R⁷ is hydrogen and each R², R⁸, R^{8a}, R⁹, R^{9a}, R¹⁰, R^{10a}, R11, and R^{11a} is deuterium. In certain embodiments, each R¹, R³, R⁴, R⁵, R⁶, R⁷, R⁹, R^{9a}, R¹⁰, R^{10a}, R11, and R^{11a} is hydrogen and each R², R⁸, and R^{8a} is deuterium. In certain embodiments, the invention provides a salt of any of the aforementioned compounds; in certain embodiments, the salt is pharmaceutically acceptable.

In certain embodiments, the compound is a compound in Table 1 below or a salt thereof.

TABLE 1

| | |
|---|---|
| Compound No. | Chemical Structure |
| I-1 | 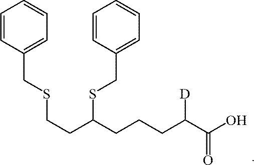 |
| I-2 | 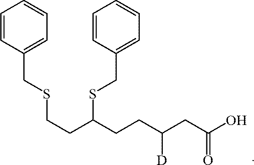 |
| I-3 | 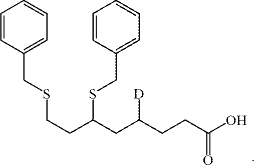 |
| I-4 | 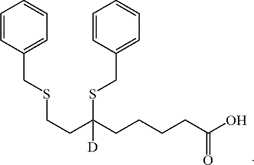 |
| 1-5 | 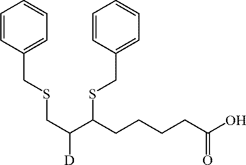 [SMILES: [2H]C(CSCc1ccccc1)C(CCCCC(=O)O)SCc1ccccc1] |
| I-6 | 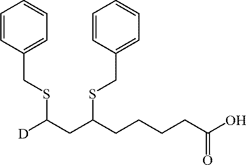 [SMILES: [2H]C(CC(CCCCC(=O)O)SCc1ccccc1)SCc1ccccc1] |
| I-7 | 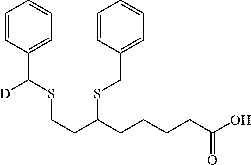 [SMILES: [2H]C(SCCC(CCCCC(=O)O)SCc1ccccc1)c1ccccc1] |
| I-8 | 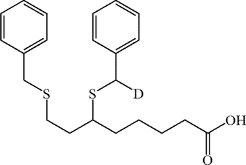 [SMILES: [2H]C(SC(CCCCC(=O)O)CCSCc1ccccc1)c1ccccc1] |
| 1-9 | 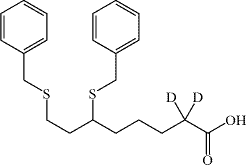 [SMILES: [2H]C([2H])(CCCC(CCSCc1ccccc1)SCc1ccccc1)C(=O)O] |
| I-10 | 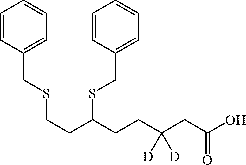 [SMILES: [2H]C([2H])(CCC(CCSCc1ccccc1)SCc1ccccc1)CC(=O)O] |
| I-11 | 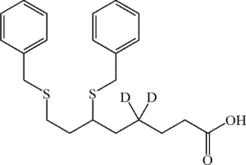 [SMILES: [2H]C([2H])(CCC(=O)O)CC(CCSCc1ccccc1)SCc1ccccc1] |
| I-12 | 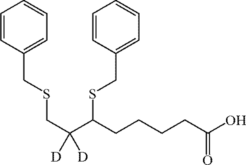 [SMILES: [2H]C([2H])(CSCc1ccccc1)C(CCCCC(=O)O)SCc1ccccc1] |
| I-13 | 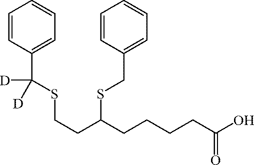 [SMILES: [2H]C([2H])(SCCC(CCCCC(=O)O)SCc1ccccc1)c1ccccc1] |
| I-14 | 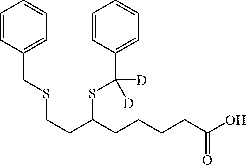 [SMILES: [2H]C([2H])(SC(CCCCC(=O)O)CCSCc1ccccc1)c1ccccc1] |
| I-15 | 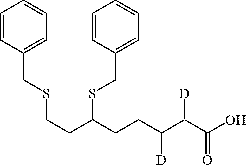 [SMILES: [2H]C(CCC(CCSCc1ccccc1)SCc1ccccc1)C([2H])C(=O)O] |
| I-16 | 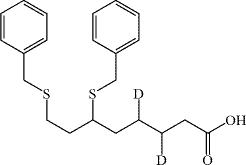 [SMILES: [2H]C(CC(=O)O)C([2H])CC(CCSCc1ccccc1)SCc1ccccc1] |
| I-17 | 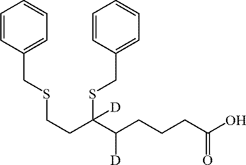 [SMILES: [2H]C(CCCC(=O)O)C([2H])(CCSCc1ccccc1)SCc1ccccc1] |
| I-18 | 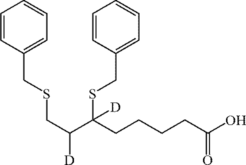 [SMILES: [2H]C(CSCc1ccccc1)C([2H])(CCCCC(=O)O)SCc1ccccc1] |
| I-19 | 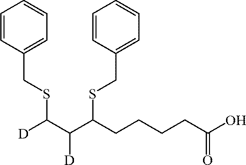 [SMILES: [2H]C(SCc1ccccc1)C([2H])C(CCCCC(=O)O)SCc1ccccc1] |
| I-20 | 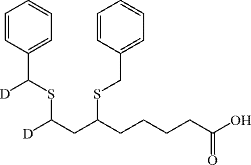 [SMILES: [2H]C(CC(CCCCC(=O)O)SCc1ccccc1)SC([2H])c1ccccc1] |
| I-21 | 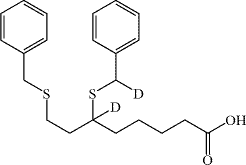 [SMILES: [2H]C(SC([2H])(CCCCC(=O)O)CCSCc1ccccc1)c1ccccc1] |
| 1-22 | 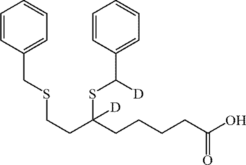 [SMILES: [2H]C(SC([2H])(CCCCC(=O)O)CCSCc1ccccc1)c1ccccc1] |
| I-23 | 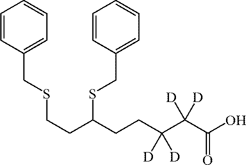 [SMILES: [2H]C([2H])(CCC(CCSCc1ccccc1)SCc1ccccc1)C([2H])([2H])C(=O)O] |
| I-24 | 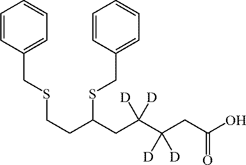 [SMILES: [2H]C([2H])(CC(=O)O)C([2H])([2H])CC(CCSCc1ccccc1)SCc1ccccc1] |
| I-25 | 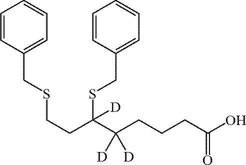 [SMILES: [2H]C([2H])(CCCC(=O)O)C([2H])(CCSCc1ccccc1)SCc1ccccc1] |
| I-26 | 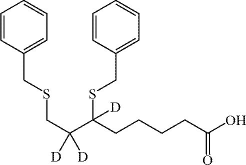 [SMILES: [2H]C([2H])(CSCc1ccccc1)C([2H])(CCCCC(=O)O)SCc1ccccc1] |
| I-27 | 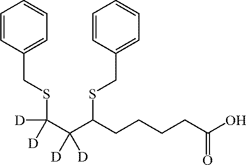 [SMILES: [2H]C([2H])(SCc1ccccc1)C([2H])([2H])C(CCCCC(=O)O)SCc1ccccc1] |
| I-28 | 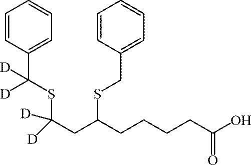 [SMILES: [2H]C([2H])(CC(CCCCC(=O)O)SCc1ccccc1)SC([2H])([2H])c1ccccc1] |
| I-29 | 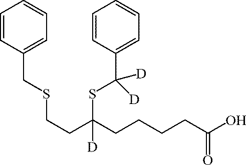 [SMILES: [2H]C(CCCCC(=O)O)(CCSCc1ccccc1)SC([2H])([2H])c1ccccc1] |
| I-30 | 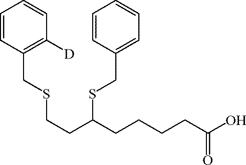 [SMILES: [2H]c1ccccc1CSCCC(CCCCC(=O)O)SCc1ccccc1] |
| I-31 | 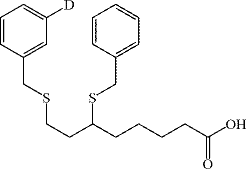 [SMILES: [2H]c1cccc(CSCCC(CCCCC(=O)O)SCc2ccccc2)c1] |
| I-32 | 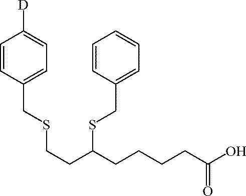 [SMILES: [2H]c1ccc(CSCCC(CCCCC(=O)O)SCc2ccccc2)cc1] |
| I-33 | 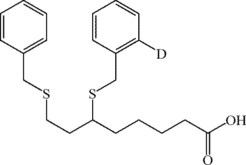 [SMILES: [2H]c1ccccc1CSC(CCCCC(=O)O)CCSCc1ccccc1] |
| I-34 | 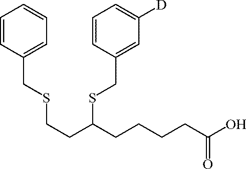 [SMILES: [2H]c1cccc(CSC(CCCCC(=O)O)CCSCc2ccccc2)c1] |
| I-35 | 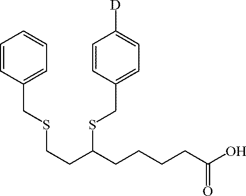 [SMILES: [2H]c1ccc(CSC(CCCCC(=O)O)CCSCc2ccccc2)cc1] |
| I-36 | 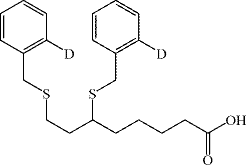 [SMILES: [2H]c1ccccc1CSCCC(CCCCC(=O)O)SCc1ccccc1[2H]] |
| I-37 | 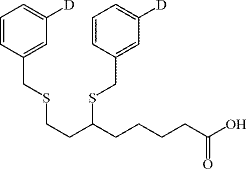 [SMILES: [2H]c1cccc(CSCCC(CCCCC(=O)O)SCc2cccc([2H])c2)c1] |
| I-38 | 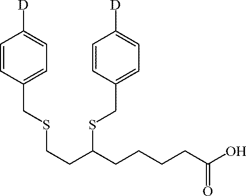 [SMILES: [2H]c1ccc(CSCCC(CCCCC(=O)O)SCc2ccc([2H])cc2)cc1] |
| I-39 | 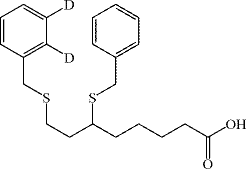 [SMILES: [2H]c1cccc(CSCCC(CCCCC(=O)O)SCc2ccccc2)c1[2H]] |
| 1-40 | 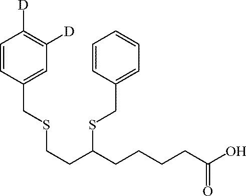 [SMILES: [2H]c1ccc(CSCCC(CCCCC(=O)O)SCc2ccccc2)cc1[2H]] |
| I-41 | 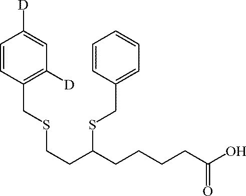 [SMILES: [2H]c1ccc(CSCCC(CCCCC(=O)O)SCc2ccccc2)c([2H])c1] |
| I-42 | 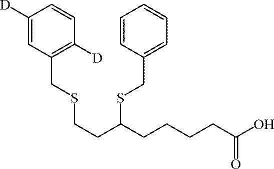 [SMILES: [2H]c1ccc([2H])c(CSCCC(CCCCC(=O)O)SCc2ccccc2)c1] |
| I-43 | 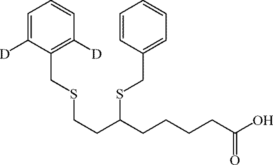 [SMILES: [2H]c1cccc([2H])c1CSCCC(CCCCC(=O)O)SCc1ccccc1] |
| I-44 | 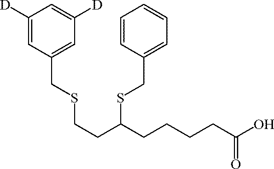 [SMILES: [2H]c1cc([2H])cc(CSCCC(CCCCC(=O)O)SCc2ccccc2)c1] |
| I-45 | 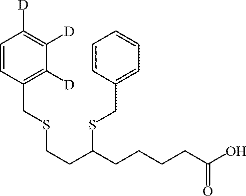 [SMILES: [2H]c1ccc(CSCCC(CCCCC(=O)O)SCc2ccccc2)c([2H])c1[2H]] |
| I-46 | 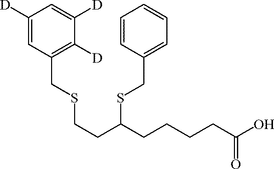 [SMILES: [2H]c1cc([2H])c([2H])c(CSCCC(CCCCC(=O)O)SCc2ccccc2)c1] |
| I-47 | 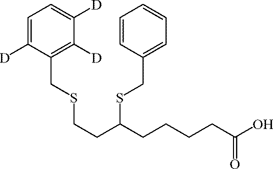 [SMILES: [2H]c1ccc([2H])c(CSCCC(CCCCC(=O)O)SCc2ccccc2)c1[2H]] |
| I-48 | 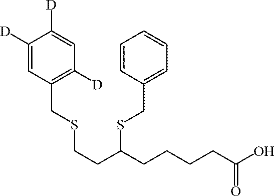 [SMILES: [2H]c1cc([2H])c(CSCCC(CCCCC(=O)O)SCc2ccccc2)cc1[2H]] |
| I-49 | 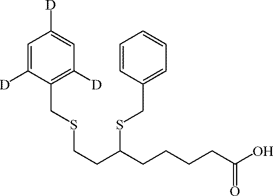 [SMILES: [2H]c1cc([2H])c(CSCCC(CCCCC(=O)O)SCc2ccccc2)c([2H])c1] |
| I-50 | 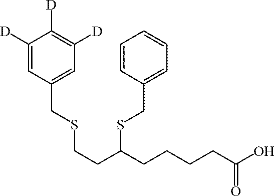 [SMILES: [2H]c1cc(CSCCC(CCCCC(=O)O)SCc2ccccc2)cc([2H])c1[2H]] |
| I-51 | 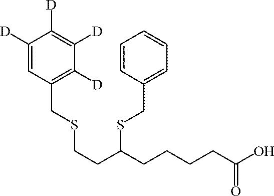 [SMILES: [2H]c1cc(CSCCC(CCCCC(=O)O)SCc2ccccc2)c([2H])c([2H])c1[2H]] |
| I-52 | 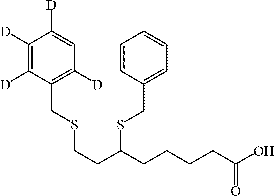 [SMILES: [2H]c1cc([2H])c(CSCCC(CCCCC(=O)O)SCc2ccccc2)c([2H])c1[2H]] |
| I-53 | 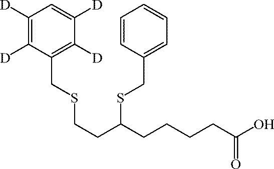 [SMILES: [2H]c1cc([2H])c([2H])c(CSCCC(CCCCC(=O)O)SCc2ccccc2)c1[2H]] |
| I-54 | 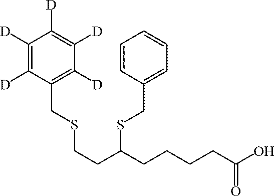 [SMILES: [2H]c1c([2H])c([2H])c(CSCCC(CCCCC(=O)O)SCc2ccccc2)c([2H])c1[2H]] |
| I-55 | 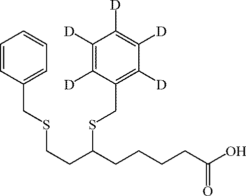 [SMILES: [2H]c1c([2H])c([2H])c(CSC(CCCCC(=O)O)CCSCc2ccccc2)c([2H])c1[2H]] |
| I-56 | 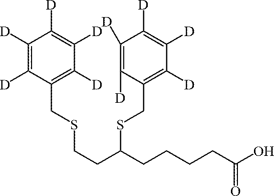 [SMILES: [2H]c1c([2H])c([2H])c(CSCCC(CCCCC(=O)O)SCc2c([2H])c([2H])c([2H])c([2H])c2[2H])c([2H])c1[2H]] |
| I-57 | 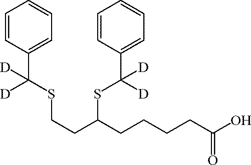 [SMILES: [2H]C([2H])(SCCC(CCCCC(=O)O)SC([2H])([2H])c1ccccc1)c1ccccc1] |
| I-58 | 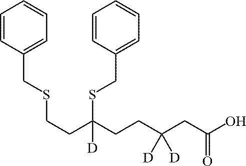 [SMILES: [2H]C([2H])(CCC([2H])(CCSCc1ccccc1)SCc1ccccc1)CC(=O)O] |
| I-59 | 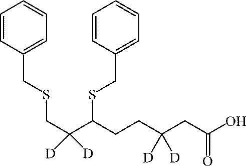 [SMILES: [2H]C([2H])(CCC(SCc1ccccc1)C([2H])([2H])CSCc1ccccc1)CC(=O)O] |
| I-60 | 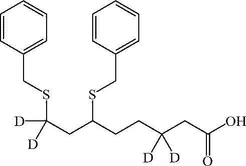 [SMILES: [2H]C([2H])(CCC(CC([2H])([2H])SCc1ccccc1)SCc1ccccc1)CC(=O)O] |
| I-61 | 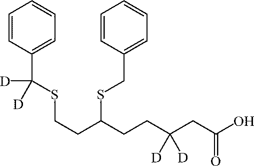 [SMILES: [2H]C([2H])(CCC(CCSC([2H])([2H])c1ccccc1)SCc1ccccc1)CC(=O)O] |
| I-62 | 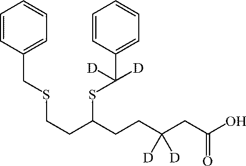 [SMILES: [2H]C([2H])(CCC(CCSCc1ccccc1)SC([2H])([2H])c1ccccc1)CC(=O)O] |
| I-63 | 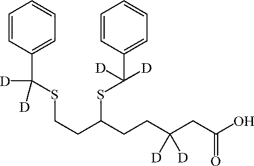 [SMILES: [2H]C([2H])(CCC(CCSC([2H])([2H])c1ccccc1)SC([2H])([2H])c1ccccc1)CC(=O)O] |
| I-64 | 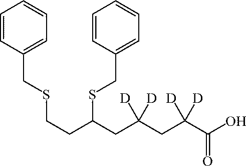 [SMILES: [2H]C([2H])(CC(CCSCc1ccccc1)SCc1ccccc1)CC([2H])([2H])C(=O)O] |
| I-65 | 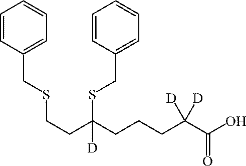 [SMILES: [2H]C(CCCC([2H])([2H])C(=O)O)(CCSCc1ccccc1)SCc1ccccc1] |
| I-66 | 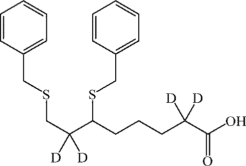 [SMILES: [2H]C([2H])(CCCC(SCc1ccccc1)C([2H])([2H])CSCc1ccccc1)C(=O)O] |
| I-67 | 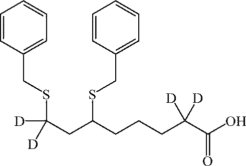 [SMILES: [2H]C([2H])(CC(CCCC([2H])([2H])C(=O)O)SCc1ccccc1)SCc1ccccc1] |
| I-68 | 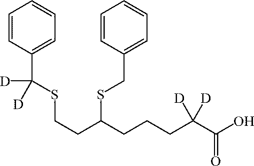 [SMILES: [2H]C([2H])(CCCC(CCSC([2H])([2H])c1ccccc1)SCc1ccccc1)C(=O)O] |
| I-69 | 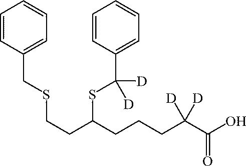 [SMILES: [2H]C([2H])(CCCC(CCSCc1ccccc1)SC([2H])([2H])c1ccccc1)C(=O)O] |
| I-70 | 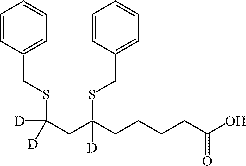 [SMILES: [2H]C([2H])(CC([2H])(CCCCC(=O)O)SCc1ccccc1)SCc1ccccc1] |
| I-71 | 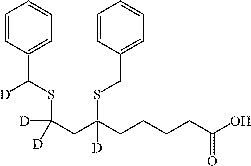 [SMILES: [2H]C(SC([2H])([2H])CC([2H])(CCCCC(=O)O)SCc1ccccc1)c1ccccc1] |
| I-72 | 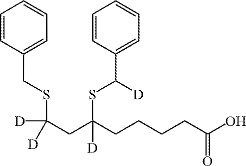 [SMILES: [2H]C(SC([2H])(CCCCC(=O)O)CC([2H])([2H])SCc1ccccc1)c1ccccc1] |
| I-73 | 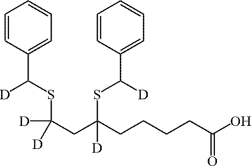 [SMILES: [2H]C(SC([2H])([2H])CC([2H])(CCCCC(=O)O)SC([2H])c1ccccc1)c1ccccc1] |
| I-74 | 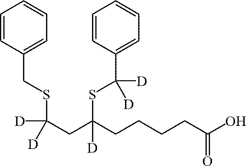 [SMILES: [2H]C([2H])(CC([2H])(CCCCC(=O)O)SC([2H])([2H])c1ccccc1)SCc1ccccc1] |
| I-75 | 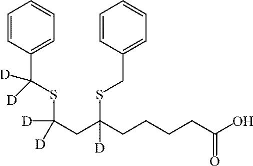 [SMILES: [2H]C([2H])(CC([2H])(CCCCC(=O)O)SCc1ccccc1)SC([2H])([2H])c1ccccc1] |
| I-76 | 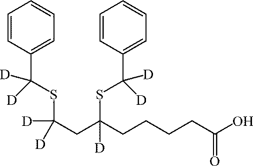 [SMILES: [2H]C([2H])(CC([2H])(CCCCC(=O)O)SC([2H])([2H])c1ccccc1)SC([2H])([2H])c1ccccc1] |
| I-77 | 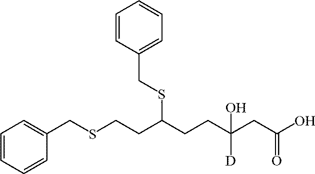 [SMILES: [2H]C(O)(CCC(CCSCc1ccccc1)SCc1ccccc1)CC(=O)O] |
| I-78 | 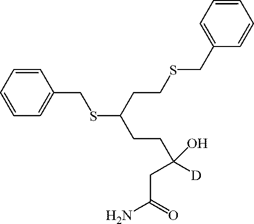 [SMILES: [2H]C(O)(CCC(CCSCc1ccccc1)SCc1ccccc1)CC(N)=O] |
| I-79 | 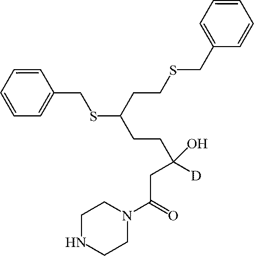 [SMILES: [2H]C(O)(CCC(CCSCc1ccccc1)SCc1ccccc1)CC(=O)N1CCNCC1] |

### III. Therapeutic Applications

The invention provides methods and compositions for treating cancer in a human patient in need thereof, comprising the step of administering to the patient a therapeutically effective amount of a compound of formula (I) or a salt thereof as described in Section II.

### Type of Cancer

In certain embodiments, the cancer is associated with altered energy metabolism. As used herein, the term “cancer” is intended to include myelodysplastic syndromes, and in certain embodiments of the present invention the cancer is a myelodysplastic syndrome. In certain embodiments, the cancer is high risk myelodysplastic syndrome (MDS). In certain embodiments, the cancer is high risk MDS in patients who have failed to respond, progressed, or relapsed while on hypomethylating therapy.

The method may be further characterized according to the severity or type of cancer. In certain embodiments, the cancer is Stage I or early stage cancer, in which the cancer is small and only in one area. In certain embodiments, the cancer is Stage II or III, in which the cancer is larger and has grown into nearby tissues or lymph nodes. In certain embodiments, the cancer is Stage IV or advanced or metastatic, in which the cancer has spread to other parts of the body.

In certain embodiments, the cancer is Stage I lymphoma, in which the cancer is found in one lymph node region or the cancer has invaded one extra-lymphatic organ or site but not any lymph node regions. In certain embodiments, the cancer is Stage II lymphoma, in which the cancer is found in two or more lymph node regions on the same side of the diaphragm or the cancer involves one organ and its regional lymph nodes, with or without cancer in other lymph node regions on the same side of the diaphragm. In certain embodiments, the cancer is Stage III lymphoma, in which there is cancer in lymph nodes on both sides of the diaphragm. In certain embodiments, the cancer is Stage IV lymphoma, in which the cancer has spread one or more organs beyond the lymph nodes.

In certain embodiments, the cancer is progressive or refractory. In certain embodiments, the cancer is a metastatic. In certain embodiments, the cancer is recurrent or relapsed. In certain embodiments, the cancer is relapsed or refractory. In certain embodiments, the cancer is a T-cell lymphoma. In certain embodiments, the cancer is a B-cell lymphoma. In certain embodiments, the cancer is previously untreated. In certain embodiments, the patient has not received hematopoietic cell transplant. In certain embodiments, the patient has received hematopoietic cell transplant.

In certain embodiments, the cancer is a lymphoma. In certain embodiments, the cancer is mantle cell lymphoma. In certain embodiments, the cancer is a leukemia. In certain embodiments, the cancer is an acute myeloid leukemia. In certain embodiments, the cancer is chronic myeloid leukemia. In certain embodiments, the cancer is acute lymphoblastic leukemia. In certain embodiments, the cancer is a carcinoma. In certain embodiments, the cancer is a sarcoma. In certain embodiments, the cancer is a myeloma. In certain embodiments, the cancer is a clear cell cancer. In certain embodiments, the cancer is a clear cell sarcoma. In certain embodiments, the cancer is a clear cell carcinoma. In certain embodiments, the cancer is a brain or spinal cord cancer. In certain embodiments, the cancer is a melanoma. In certain embodiments, the cancer is a blastoma. In certain embodiments, the cancer is a germ cell tumor. In certain embodiments, the cancer is a cancer of the pancreas. In certain embodiments, the cancer is a metastatic pancreatic cancer. In certain embodiments, the cancer is a locally advanced pancreatic cancer. In certain embodiments, the cancer is a cancer of the prostate. In certain embodiments, the cancer is a castration resistant prostate cancer. In certain embodiments, the cancer is a cancer of the lung. In certain embodiment, the cancer is non-small cell lung cancer. In certain embodiments, the cancer is a cancer of the colon. In certain embodiments, the cancer is a cancer of the rectum. In certain embodiments, the cancer is a colorectal cancer. In certain embodiments, the cancer is a cancer of the cervix. In certain embodiments, the cancer is a neuroendocrine tumor. In certain embodiments, the cancer is a gastroenteropancreatic neuroendocrine tumor. In certain embodiments, the cancer is a cancer of the liver. In certain embodiments, the cancer is a cancer of the uterus. In certain embodiments, the cancer is a cancer of the cervix. In certain embodiments, the cancer is a cancer of the bladder. In certain embodiments, the cancer is a cancer of the kidney. In certain embodiments, the cancer is a cancer of the breast. In certain embodiments, the cancer is a cancer of the ovary. In certain embodiments, the cancer is a cancer of the biliary tract.

In certain embodiments, the cancer is Burkitt’s Lymphoma. In certain embodiments, the cancer is relapsed or refractory Burkitt’s Lymphoma. In certain embodiments, the cancer is relapsed or refractory Burkitt’s Lymphoma in which the patient has failed at least one previous line of therapy. In certain embodiments, the cancer is relapsed or refractory Burkitt’s Lymphoma in which the patient has failed prior bone marrow transplant. In certain embodiments, the cancer is double hit diffuse large B cell lymphoma. In certain embodiments, the cancer is high-grade B cell lymphoma with rearrangements of MYC and BCL2 and/or BCL6 (DHL/THL). In certain embodiments, the cancer is Hodgkin lymphoma. In certain embodiments, the cancer is non-Hodgkin lymphoma. In certain embodiments, the cancer is T-cell non-Hodgkin lymphoma. In certain embodiments, the cancer is relapsed or refractory Hodgkin lymphoma. In certain embodiments, the cancer is relapsed or refractory non-Hodgkin lymphoma. In certain embodiments, the cancer is relapsed or refractory T-cell non-Hodgkin lymphoma. In certain embodiments, the cancer is Hodgkin lymphoma in which the patient has not received hematopoietic cell transplant. In certain embodiments, the cancer is Hodgkin lymphoma in which the patient has received hematopoietic cell transplant. In certain embodiments, the cancer is non-Hodgkin lymphoma in which the patient has not received hematopoietic cell transplant. In certain embodiments, the cancer is non-Hodgkin lymphoma in which the patient has received hematopoietic cell transplant. In certain embodiments, the cancer is T-cell non-Hodgkin lymphoma in which the patient has not received hematopoietic cell transplant. In certain embodiments, the cancer is T-cell non-Hodgkin lymphoma in which the patient has received hematopoietic cell transplant. In certain embodiments, the cancer is relapsed or refractory Hodgkin lymphoma in which the patient has not received hematopoietic cell transplant. In certain embodiments, the cancer is relapsed or refractory Hodgkin lymphoma in which the patient has received hematopoietic cell transplant. In certain embodiments, the cancer is relapsed or refractory non-Hodgkin lymphoma in which the patient has not received hematopoietic cell transplant. In certain embodiments, the cancer is relapsed or refractory Hodgkin lymphoma in which the patient has or has not received hematopoietic cell transplant. In certain embodiments, the cancer is relapsed or refractory Hodgkin lymphoma in which the patient has failed brentuximab vedotin and a PD-1 inhibitor. In certain embodiments, the cancer is relapsed or refractory Hodgkin lymphoma in which the patient has failed brentuximab vedotin and a PD-1 inhibitor and has received hematopoietic cell transplant. In certain embodiments, the cancer is relapsed or refractory Hodgkin lymphoma in which the patient has failed brentuximab vedotin and a PD-1 inhibitor and has not received hematopoietic cell transplant. In certain embodiments, the cancer is relapsed or refractory non-Hodgkin lymphoma in which the patient has received hematopoietic cell transplant. In certain embodiments, the cancer is relapsed or refractory T-cell non-Hodgkin lymphoma in which the patient has not received hematopoietic cell transplant. In certain embodiments, the cancer is relapsed or refractory T-cell non-Hodgkin lymphoma in which the patient has received hematopoietic cell transplant. In certain embodiments, the cancer is relapsed or refractory T-cell non-Hodgkin lymphoma in which the patient has or has not received hematopoietic cell transplant.

### General Aspects of Administering a Therapeutic Agent to a Patient

Generally, the compound of formula (I) or a pharmaceutically acceptable salt thereof as described in Section II is delivered to the patient in a therapeutically effective amount, sufficient to treat cancer. The treatment may involve one or several administrations on one or more days, and the dosage may be adjusted by the individual practitioner to achieve a desired effect. Preferably, the dosage amount should be sufficient to interact primarily with disease cells, leaving normal cells comparatively unharmed.

The dosage amount may be administered in a single dose or in the form of individual divided doses, such as one, two, three, or four times per day. In certain embodiments, the daily dosage amount is administered in a single dose. In the event that the response in a patient is insufficient at a certain dose, higher or more frequent doses may be employed to the extent of patient tolerance.

For combination therapy, each agent may be administered in a particular order and/or on the same or different days according to a treatment cycle. For example, a dose of a compound of formula (I) or a pharmaceutically acceptable salt thereof as described in Section II may be administered to the patient prior to administering a second therapeutic agent, such as immediately prior, earlier in the day, or on an earlier day in a treatment cycle. In certain embodiments, the active agents may be administered on the same day of a treatment cycle, for example being co-administered simultaneously or one right after the other. In certain embodiments, a dose of a second therapeutic agent is administered to the patient prior to administering the compound of formula (I) or a pharmaceutically acceptable salt thereof, such as immediately prior, earlier in the day, or on an earlier day in a treatment cycle. In certain embodiments, treatment cycles may be repeated one or more times in order to maximize benefit to the patient.

### Compound of Formula (I)

The compound of formula (I) as described in Section II may be administered in any suitable form, including as a solid or liquid, a free acid or salt. The compound of formula (I) may be crystalline, amorphous, or dissolved in solution. In certain embodiments, the compound of formula (I) is administered to the patient as a salt or ion pair. In certain embodiments, the compound of formula (I) is administered to the patient as a salt or ion pair with triethanolamine. Exemplary ion pairing agents that may be used include, for example, a tertiary amine (such as triethylamine or triethanolamine), other amines such as diethylamine, diethanolamine, monoethanolamine, meglumine, mefenamic acid and tromethamine, and combinations thereof. In certain embodiments, the ion pairing agent is an organic Bronsted base. In certain other embodiments, the ion pairing agent is an amine compound. In yet other embodiments, the ion pairing agent is a monoalkylamine, dialkylamine, trialkylamine, amino-substituted aliphatic alcohol, hydroxymonoalkylamine, hydroxydialkylamine, hydroxytrialkylamine, amino-substituted heteroaliphatic alcohol, alkyldiamine, substituted alkyldiamine, or optionally substituted heteroaryl group containing at least one ring nitrogen atom. In certain embodiments, the therapeutic agent is a salt of a compound of formula (I) as described above with an ion pairing agent selected with guidance from Berge et al., “Pharmaceutical Salts,” J. of Pharmaceutical Science, 1977; 66:1-19 or Handbook of Pharmaceutical Salts Properties, Selection and Use, IUPAC, Wiley-VCH, P. H. Stahl, ed., the entire disclosures of which are incorporated by reference herein. Ion pairing agents of particular note in the latter include, without limitation, those listed in Table 5, p. 342.

Additional exemplary ion pairing agents include, for example, polyethyleneimine, polyglutamic acid, ammonia, L-arginine, benethamine benzathine, betaine, calcium hydroxide, choline, deanol, diethanolamine(2,2′-iminobis(ethanol)), diethylamine, 2-(diethylamino)-ethanol, ethanolamine, ethylenediamine, N-methyl-glucamine, hydrabamine, 1H-imidazole, lysine, magnesium hydroxide, 4-(2-hydroxyethyl)-morpholine, piperazine, potassium hydroxide, 1-(2-hydroxyethyl)-pyrrolidine, sodium hydroxide, triethanolamine (2,2′,2″-nitrilotris(ethanol)), tromethamine, and zinc hydroxide. In certain other embodiments, the ion pairing agent is diisopropanolamine, 3-amino-1-propanol, meglumine, morpholine, pyridine, niacinamide, tris(hydroxymethyl)aminomethane, 2-((2-dimethylamino)ethoxy)ethanol, 2-(dimethylamino)ethanol, 1-(2-hydroxyethyl)pyrrolidine, or ammonium hydroxide. In certain other embodiments, the ion pairing agent is an alkali metal hydroxide or alkaline earth metal hydroxide, such as, for example, cesium hydroxide.

### Route of Administration

The compound of formula (I) or pharmaceutically acceptable salt thereof as described in Section II may be administered to the patient by any suitable route. For example, in certain embodiments, the compound of formula (I) or pharmaceutically acceptable salt thereof is administered orally to the patient. In certain embodiments, the compound of formula (1) or pharmaceutically acceptable salt thereof is administered subcutaneously to the patient. In certain embodiments, the compound of formula (I) or pharmaceutically acceptable salt thereof is administered intravenously to the patient. In certain embodiments, the compound of formula (I) or pharmaceutically acceptable salt thereof is administered as an IV infusion over two hours. In certain embodiments, the compound of formula (I) or pharmaceutically acceptable salt thereof is administered as an IV infusion over two hours via a central venous catheter. An advantage of the present invention is that the compound of formula (I) or pharmaceutically acceptable salt thereof may be administered at a lower dose than the corresponding dose of 6,8-bis(benzylsulfanyl)octanoic acid for a particular indication. When administered by IV infusion, the lower dose may permit decreased infusion times as compared to 6,8-bis(benzylsulfanyl)octanoic acid. When administered orally, the lower dose may permit smaller and/or fewer tablets or capsules per dose as compared to 6,8-bis(benzylsulfanyl)octanoic acid.

### Pharmaceutical Composition

One aspect of the invention provides pharmaceutical compositions. The pharmaceutical composition desirably comprises a compound described herein, such as a compound of formula (I) and a pharmaceutically acceptable carrier.

Any suitable pharmaceutical composition may be used to administer the compound of formula (I) or pharmaceutically acceptable salt thereof as described in Section II to the patient. In combination therapy, the therapeutic agents may be administered together in the same pharmaceutical composition (e.g., fixed dose combination) or separately in different pharmaceutical compositions. There is a wide variety of suitable formulations of pharmaceutical compositions of the present invention (see, e.g., Remington: The Science and Practice of Pharmacy, 20th ed., Gennaro et al. Eds., Lippincott Williams and Wilkins, 2000). In certain embodiments, the compound of formula (I) or a pharmaceutically acceptable salt thereof as described in Section II is administered in a pharmaceutical composition that is a dry oral dosage form. In certain embodiments, the pharmaceutical composition is an oral dosage form chosen from tablet, pill, capsule, caplet, powder, granule, solution, suspension, and gel. Oral dosage forms may include pharmaceutically acceptable excipients, such as carriers, diluents, stabilizers, plasticizers, binders, glidants, disintegrants, bulking agents, lubricants, plasticizers, colorants, film formers, flavoring agents, preservatives, dosing vehicles, and any combination of any of the foregoing.

The pharmaceutical composition will generally include at least one inert excipient. Excipients include pharmaceutically compatible binding agents, lubricants, wetting agents, disintegrants, and the like. Tablets, pills, capsules, troches and the like can contain any of the following excipients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring. When the dosage unit form is a capsule, it can contain a liquid excipient such as a fatty oil. In addition, dosage unit forms can contain various other materials that modify the physical form of the dosage unit, for example, coatings of sugar, shellac, or enteric agents. Further, a syrup may contain, in addition to the active compounds, sucrose as a sweetening agent and certain preservatives, dyes, colorings, and flavorings. In certain embodiments, the pharmaceutical composition comprises an excipient in an amount of about 5% to about 99%, such as about 10% to about 85%, by weight of the composition, with the therapeutic agent comprising the remainder. In certain embodiments, pharmaceutically acceptable excipients comprise about 20% to about 80% of the total weight of the composition. In certain embodiments, the pharmaceutical composition comprises the therapeutic agent in an amount of at least about 40% by weight of the composition, with one or more excipients comprising the remainder. In certain embodiments, the pharmaceutical composition comprises the therapeutic agent in an amount of at least about 50% by weight of the composition. In certain embodiments, the pharmaceutical composition comprises the therapeutic agent in an amount of at least about 60% by weight of the composition. In certain embodiments, the pharmaceutical composition comprises the therapeutic agent in an amount of at least about 70% by weight of the composition. In certain embodiments, the pharmaceutical composition comprises the therapeutic agent in an amount of at least about 80% by weight of the composition. In certain embodiments, the pharmaceutical composition comprises the therapeutic agent in an amount of at least about 90% by weight of the composition.

Diluents for solid compositions include, but are not limited to, microcrystalline cellulose (e.g. AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit), potassium chloride, powdered cellulose, sodium chloride, sorbitol and talc.

Binders for solid pharmaceutical compositions include, but are not limited to, acacia, tragacanth, sucrose, glucose, alginic acid, carbomer (e.g. Carbopol), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate and starch. In certain embodiments, the pharmaceutical composition comprises a binder in an amount of about 0.5% to about 25%, such as about 0.75% to about 15%, by weight of the composition. In certain embodiments, the pharmaceutical composition comprises a binder in an amount of about 1% to about 10% by weight of the composition.

The dissolution rate of a compacted solid pharmaceutical composition in a patient’s stomach may be increased by the addition of a disintegrant to the composition. Disintegrants include, but are not limited to, alginic acid, carboxymethylcellulose calcium, carboxymethylcellulose sodium (e.g. AC-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, powdered cellulose, pregelatinized starch, sodium alginate, sodium starch glycolate (e.g. EXPLOTAB®) and starch. In certain embodiments, the pharmaceutical composition comprises a disintegrant in an amount of about 0.2% to about 30%, such as about 0.2% to about 10%, by weight of the composition. In certain embodiments, the pharmaceutical composition comprises a disintegrant in an amount of about 0.2% to about 5% by weight of the composition.

The pharmaceutical composition optionally comprises one or more pharmaceutically acceptable wetting agents. Such wetting agents are preferably selected to maintain the API in close association with water, a condition that is believed to improve bioavailability of the composition. Non-limiting examples of surfactants that can be used as wetting agents include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene, caprylic/capric mono- and diglycerides (e.g., Labrasol™ of Gattefosse), polyoxyethylene castor oil and polyoxyethylene hydrogenated castor oil; polyoxyethylene alkyl ethers, for example polyoxyethylene cetostearyl ether, polyoxyethylene fatty acid esters, for example polyoxyethylene stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefosse), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. In certain embodiments, the pharmaceutical composition comprises a wetting agent in an amount of about 0.25% to about 15%, such as about 0.4% to about 10%, by weight of the composition. In certain embodiments, the pharmaceutical composition comprises a wetting agent in an amount of about 0.5% to about 5% by weight of the composition. In certain embodiments, the pharmaceutical composition comprises a wetting agent that is an anionic surfactant. In certain embodiments, the pharmaceutical composition comprises sodium lauryl sulfate as a wetting agent. In certain embodiments, the pharmaceutical composition comprises sodium lauryl sulfate in an amount of about 0.25% to about 7%, such as about 0.4% to about 4%, by weight of the composition. In certain embodiments, the pharmaceutical composition comprises sodium lauryl sulfate in an amount of about 0.5% to about 2% by weight of the composition.

Lubricants (e.g., anti-adherents or glidants) can be added to improve the flow properties of solid compositions and/or to reduce friction between the composition and equipment during compression of tablet formulations. Excipients that may function as lubricants include, but are not limited to, colloidal silicon dioxide, magnesium trisilicate, powdered cellulose, starch, talc and tribasic calcium phosphate. Suitable lubricants further include glyceryl behapate (e.g., Compritol™ 888 of Gattefosse); stearic acid and salts thereof, including magnesium, calcium and sodium stearates; zinc stearate; glyceryl monostearate; glyceryl palmitostearate; hydrogenated castor oil; hydrogenated vegetable oils (e.g., Sterotex™ of Abitec); waxes; boric acid; sodium benzoate; sodium acetate; sodium stearyl fumarate; sodium fumarate; sodium chloride; DL-leucine; PEG (e.g., Carbowax™ 4000 and Carbowax™ 6000 of the Dow Chemical Company); sodium oleate; sodium lauryl sulfate; and magnesium lauryl sulfate. In certain embodiments, the pharmaceutical composition comprises a lubricant in an amount of about 0.1% to about 10%, such as about 0.2% to about 8%, by weight of the composition. In certain embodiments, the pharmaceutical composition comprises a lubricant in an amount of about 0.25% to about 5% by weight of the composition. In certain embodiments, the pharmaceutical composition comprises magnesium stearate as a lubricant. In certain embodiments, the pharmaceutical composition comprises colloidal silicon dioxide. In certain embodiments, the pharmaceutical composition comprises talc. In certain embodiments, the composition comprises magnesium stearate or talc in an amount of about 0.5% to about 2% by weight of the composition.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid ethyl maltol, and tartaric acid.

Compositions may also be colored using any pharmaceutically acceptable colorant to improve their appearance and/or facilitate patient identification of the product and unit dosage level. The formulations of the invention may be buffered by the addition of suitable buffering agents.

In certain embodiments of the present invention, the compound of formula (I) or a pharmaceutically salt thereof as described in Section II may be formulated as a pharmaceutically-acceptable oil; liposome; oil-water or lipid-oil-water emulsion or nanoemulsion; or liquid. To facilitate such formulations, the compound of formula (I) or pharmaceutically acceptable salt thereof may be combined with a pharmaceutically-acceptable excipient therefor.

As described in detail below, the pharmaceutical compositions may be specially formulated for administration in solid or liquid form, including those adapted for parenteral administration, for example, by subcutaneous, intramuscular, intravenous or epidural injection as, for example, a sterile solution or suspension, or sustained-release formulation.

Further examples of pharmaceutical formulations suitable for administration of a compound of formula (I) or a pharmaceutically salt thereof are described in U.S. Pat. No. 8,263,653, the entire disclosure of which is incorporated by reference herein.

Methods of preparing pharmaceutical formulations or pharmaceutical compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

In certain embodiments, one or more of the therapeutic agents are administered by intraparenteral administration. In certain other embodiments, one or more of the therapeutic agents are formulated for inhalational, oral, topical, transdermal, nasal, ocular, pulmonary, rectal, transmucosal, intravenous, intramuscular, subcutaneous, intraperitoneal, intrathoracic, intrapleural, intrauterine, intratumoral, or infusion methodologies or administration, or combinations of any thereof, in the form of aerosols, sprays, powders, gels, lotions, creams, suppositories, ointments, and the like. As indicated above, if such a formulation is desired, other additives known in the art may be included to impart the desired consistency and other properties to the formulation.

In certain embodiments, the pharmaceutical composition of the present invention is a unit dose composition. In certain embodiments, the pharmaceutical composition contains about 1 mg to about 5000 mg of the compound of formula (I) or a pharmaceutically salt thereof. In certain embodiments, the pharmaceutical composition contains about 100 mg to about 3000 mg of the compound of formula (I) or a pharmaceutically salt thereof. In certain embodiments, the pharmaceutical composition contains about 200 mg to about 2000 mg of the compound of formula (I) or a pharmaceutically salt thereof. In certain embodiments, the pharmaceutical composition contains about 50 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2500 mg, or 3000 mg of the compound of formula (I) or a pharmaceutically salt thereof. In certain embodiments, the pharmaceutical composition contains about 300 mg, 500 mg, 700 mg, or 1000 mg of the compound of formula (I) or a pharmaceutically salt thereof.

In certain embodiments, the pharmaceutical composition of the present invention comprises an emulsion, particle, or gel as described in U.S. Pat. No. 7,220,428. In certain embodiments, the pharmaceutical composition is a solid or liquid formulation having from about 0.1% to about 75% w/w lipids or fatty acid components. In certain embodiments, the formulation contains about 0.1% to about 15% w/v lipids and fatty acid components. In certain embodiments, the fatty acid component comprises saturated or unsaturated C4, C5, C6, C7, C8, C9, C10, C11, or C12 fatty acids and/or salts of such fatty acids. Lipids may include cholesterol and analogs thereof.

In certain embodiments, the pharmaceutical composition of the compound of formula (I) comprises triethanolamine and compound of formula (I) in a mole ratio of triethanolamine to compound of formula (I) of about 10:1 to about 1:10. In certain embodiments, the mole ratio of triethanolamine to compound of formula (I) is about 10:1 to about 5:1. In certain embodiments, the mole ratio of triethanolamine to compound of formula (I) is about 8:1. In certain embodiments, the pharmaceutical composition comprises a 50 mg/mL solution of a compound of formula (I) in 1 M aqueous triethanolamine. In certain embodiments, the pharmaceutical composition comprises a solution of a compound of formula (I) in 1 M aqueous triethanolamine diluted from 50 mg/mL to as low as 12.5 mg/mL with sterile aqueous 5% dextrose for injection (D5W). In certain embodiments, the pharmaceutical composition comprises a solution of a compound of formula (I) in 1 M aqueous triethanolamine diluted from 50 mg/mL to about 12.5 mg/mL with sterile aqueous 5% dextrose for injection (D5W).

### Dosing Amounts & Schedules

The compound of formula (I) or a pharmaceutically salt thereof as described in Section II may be administered to the patient in a therapeutically effective dose according to any suitable schedule. The therapeutically effective dose and schedule will vary based on the cancer being treated and can be readily determined by those of ordinary skill in the art in view of the 6,8-bis(benzylsulfanyl)octanoic acid doses and schedules used in the prior art when administered alone or in combination with other agents, as well as the guidance provided herein. In certain embodiments, the dose is the maximum tolerated dose. An advantage of the present invention is that the compound of formula (I) may be administered at a lower dose than 6,8-bis(benzylsulfanyl)octanoic acid while achieving the same or similar exposure. Preferably, the lower dose provides the same or similar efficacy with the same or lower side effects.

In certain embodiments, the compound of formula (I) or a pharmaceutically salt thereof is administered at a daily dose of about 25 mg/m² to about 5000 mg/m². In certain embodiments, the compound of formula (I) or a pharmaceutically salt thereof is administered at a daily dose of about 50 mg/m² to about 4000 mg/m². In certain embodiments, the first compound of formula (I) or a pharmaceutically salt thereof is administered at a daily dose of about 100 mg/m² to about 3000 mg/m². In certain embodiments, the compound of formula (I) or a pharmaceutically salt thereof is administered at a daily dose of about 150 mg/m² to about 3000 mg/m². In certain embodiments, the compound of formula (I) or a pharmaceutically salt thereof is administered at a daily dose of about 250 mg/m² to about 2500 mg/m². In certain embodiments, the first compound of formula (I) or a pharmaceutically salt thereof is administered at a daily dose of about 500 mg/m² to about 2000 mg/m². In certain embodiments, the compound of formula (I) or a pharmaceutically salt thereof is administered at a daily dose of about 25 mg/m². In certain embodiments, the compound of formula (I) or a pharmaceutically salt thereof is administered at a daily dose of about 500 mg/m². In certain embodiments, the compound of formula (I) or a pharmaceutically salt thereof is administered at a daily dose of about 100 mg/m². In certain embodiments, the compound of formula (I) or a pharmaceutically salt thereof is administered at a daily dose of about 150 mg/m². In certain embodiments, the compound of formula (I) or a pharmaceutically salt thereof is administered at a daily dose of about 200 mg/m². In certain embodiments, the compound of formula (I) or a pharmaceutically salt thereof is administered at a daily dose of about 250 mg/m². In certain embodiments, the compound of formula (I) or a pharmaceutically salt thereof is administered at a daily dose of about 300 mg/m². In certain embodiments, the compound of formula (I) or a pharmaceutically salt thereof is administered at a daily dose of about 350 mg/m². In certain embodiments, the compound of formula (I) or a pharmaceutically salt thereof is administered at a daily dose of about 400 mg/m². In certain embodiments, the compound of formula (I) or a pharmaceutically salt thereof is administered at a daily dose of about 450 mg/m². In certain embodiments, the compound of formula (I) or a pharmaceutically salt thereof is administered at a daily dose of about 500 mg/m². In certain embodiments, the compound of formula (I) or a pharmaceutically salt thereof is administered at a daily dose of about 600 mg/m². In certain embodiments, the compound of formula (I) or a pharmaceutically salt thereof is administered at a daily dose of about 700 mg/m². In certain embodiments, the compound of formula (I) or a pharmaceutically salt thereof is administered at a daily dose of about 800 mg/m². In certain embodiments, the compound of formula (I) or a pharmaceutically salt thereof is administered at a daily dose of about 900 mg/m². In certain embodiments, the compound of formula (I) or a pharmaceutically salt thereof is administered at a daily dose of about 1000 mg/m². In certain embodiments, the compound of formula (I) or a pharmaceutically salt thereof is administered at a daily dose of about 1100 mg/m². In certain embodiments, the compound of formula (I) or a pharmaceutically salt thereof is administered at a daily dose of about 1200 mg/m². In certain embodiments, the compound of formula (I) or a pharmaceutically salt thereof is administered at a daily dose of about 1300 mg/m². In certain embodiments, the compound of formula (I) or a pharmaceutically salt thereof is administered at a daily dose of about 1400 mg/m². In certain embodiments, the compound of formula (I) or a pharmaceutically salt thereof is administered at a daily dose of about 1500 mg/m². In certain embodiments, the compound of formula (I) or a pharmaceutically salt thereof is administered at a daily dose of about 1600 mg/m². In certain embodiments, the compound of formula (I) or a pharmaceutically salt thereof is administered at a daily dose of about 1700 mg/m². In certain embodiments, the compound of formula (I) or a pharmaceutically salt thereof is administered at a daily dose of about 1800 mg/m². In certain embodiments, the compound of formula (I) or a pharmaceutically salt thereof is administered at a daily dose of about 1900 mg/m². In certain embodiments, the compound of formula (I) or a pharmaceutically salt thereof is administered at a daily dose of about 2000 mg/m². In certain embodiments, the compound of formula (I) or a pharmaceutically salt thereof is administered at a daily dose of about 2500 mg/m². In certain embodiments, the compound of formula (I) or a pharmaceutically salt thereof is administered at a daily dose of about 3000 mg/m².

In certain embodiments, the compound of formula (I) or a pharmaceutically salt thereof is administered at a daily dose of about 1 mg to about 10,000 mg. In certain embodiments, the compound of formula (I) or a pharmaceutically salt thereof is administered at a daily dose of about 10 mg to about 7,500 mg. In certain embodiments, the compound of formula (I) or a pharmaceutically salt thereof is administered at a daily dose of about 100 mg to about 5,000 mg. In certain embodiments, the compound of formula (I) or a pharmaceutically salt thereof is administered at a daily dose of about 200 mg to about 4,000 mg. In certain embodiments, the compound of formula (I) or a pharmaceutically salt thereof is administered at a daily dose of about 300 mg to about 3,000 mg. In certain embodiments, the compound of formula (I) or a pharmaceutically salt thereof is administered at a daily dose of about 400 mg to about 2,500 mg. In certain embodiments, the compound of formula (I) or a pharmaceutically salt thereof is administered at a daily dose of about 500 mg to about 2,000 mg. In certain embodiments, the compound of formula (I) or a pharmaceutically salt thereof is administered at a daily dose of about 100 mg. In certain embodiments, the compound of formula (I) or a pharmaceutically salt thereof is administered at a daily dose of about 200 mg. In certain embodiments, the compound of formula (I) or a pharmaceutically salt thereof is administered at a daily dose of about 300 mg. In certain embodiments, the compound of formula (I) or a pharmaceutically salt thereof is administered at a daily dose of about 400 mg. In certain embodiments, the compound of formula (I) or a pharmaceutically salt thereof is administered at a daily dose of about 500 mg. In certain embodiments, the compound of formula (I) or a pharmaceutically salt thereof is administered at a daily dose of about 600 mg. In certain embodiments, the compound of formula (I) or a pharmaceutically salt thereof is administered at a daily dose of about 700 mg. In certain embodiments, the compound of formula (I) or a pharmaceutically salt thereof is administered at a daily dose of about 800 mg. In certain embodiments, the compound of formula (I) or a pharmaceutically salt thereof is administered at a daily dose of about 900 mg. In certain embodiments, the compound of formula (I) or a pharmaceutically salt thereof is administered at a daily dose of about 1,000 mg. In certain embodiments, the compound of formula (I) or a pharmaceutically salt thereof is administered at a daily dose of about 1,250 mg. In certain embodiments, the compound of formula (I) or a pharmaceutically salt thereof is administered at a daily dose of about 1,500 mg. In certain embodiments, the compound of formula (I) or a pharmaceutically salt thereof is administered at a daily dose of about 1,750 mg. In certain embodiments, the compound of formula (I) or a pharmaceutically salt thereof is administered at a daily dose of about 2,000 mg. In certain embodiments, the compound of formula (I) or a pharmaceutically salt thereof is administered at a daily dose of about 2,500 mg. In certain embodiments, the compound of formula (I) or a pharmaceutically salt thereof is administered at a daily dose of about 3,000 mg. In certain embodiments, the compound of formula (I) or a pharmaceutically salt thereof is administered at a daily dose of about 3,500 mg. In certain embodiments, the compound of formula (I) or a pharmaceutically salt thereof is administered at a daily dose of about 4,000 mg. In certain embodiments, the compound of formula (I) or a pharmaceutically salt thereof is administered at a daily dose of about 4,500 mg. In certain embodiments, the compound of formula (I) or a pharmaceutically salt thereof is administered at a daily dose of about 5,000 mg. In certain embodiments, the compound of formula (I) or a pharmaceutically salt thereof is administered at a daily dose of about 6,000 mg. In certain embodiments, the compound of formula (I) or a pharmaceutically salt thereof is administered at a daily dose of about 7,000 mg. In certain embodiments, the compound of formula (I) or a pharmaceutically salt thereof is administered at a daily dose of about 8,000 mg. In certain embodiments, the compound of formula (I) or a pharmaceutically salt thereof is administered at a daily dose of about 9,000 mg. In certain embodiments, the compound of formula (I) or a pharmaceutically salt thereof is administered at a daily dose of about 10,000 mg.

The daily dose of compound of formula (I) or a pharmaceutically salt thereof may be administered as one dose or divided into two or more doses - e.g., b.i.d. (two times a day), t.i.d. (three times a day), or q.i.d. (four times a day). At higher daily doses and/or when administered orally or subcutaneously, it will often be beneficial to administer the daily dose of compound of formula (I) or a pharmaceutically salt thereof b.i.d., t.i.d., or q.i.d. Splitting the daily dose may improve efficacy by prolonging exposure time and may also improve safety by reducing peak plasma concentration.

The compound of formula (I) or a pharmaceutically salt thereof may be administered pursuant to a treatment schedule that includes days in which a dose of compound of formula (I) or a pharmaceutically salt thereof is administered and days in which a dose of compound of formula (I) or a pharmaceutically salt thereof is not administered. For example, the compound of formula (I) or a pharmaceutically salt thereof may be administered pursuant to a schedule in which compound of formula (I) or a pharmaceutically salt thereof is administered during the early days of a cycle and then not administered during the latter portion of the cycle. In certain embodiments, the compound of formula (I) or a pharmaceutically salt thereof is administered on days 1-5 of a 28-day cycle. In certain embodiments, the compound of formula (I) or a pharmaceutically salt thereof is administered on days 1, 8, and 15 of a four-week cycle. In certain embodiments, the compound of formula (I) or a pharmaceutically salt thereof is administered on days 1 and 3 of a two-week cycle. In certain embodiments, the compound of formula (I) or a pharmaceutically salt thereof is administered on days 1-5 of a three-week cycle. In certain embodiments, the compound of formula (I) or a pharmaceutically salt thereof is administered on days 1-5 of a two-week cycle. In certain embodiments, the compound of formula (I) or a pharmaceutically salt thereof is administered on days 1-3 of a three-week cycle. In certain embodiments, the compound of formula (I) or a pharmaceutically salt thereof is administered on days 1-3 of a two-week cycle. In certain embodiments, the compound of formula (I) or a pharmaceutically salt thereof is administered every day. In certain embodiments, the compound of formula (I) or a pharmaceutically salt thereof is administered every other day. In certain embodiments, the compound of formula (I) or a pharmaceutically salt thereof is administered three days per week. In certain embodiments, the compound of formula (I) or a pharmaceutically salt thereof is administered two days per week. In certain embodiments, the compound of formula (I) or a pharmaceutically salt thereof is administered one day per week.

In certain embodiments, the dosing cycle is repeated at least once. In certain embodiments, the method of the present invention comprises treatment with 5 cycles or more. In certain embodiments, the method of the present invention comprises treatment with 6 cycles or more. In certain embodiments, the method of the present invention comprises treatment with 7 cycles or more. In certain embodiments, the method of the present invention comprises treatment with 8 cycles or more. In certain embodiments, the method of the present invention comprises treatment with 9 cycles or more. In certain embodiments, the method of the present invention comprises treatment with 10 cycles or more.

### Patients for Treatment

The therapeutic methods may be further characterized according to the patient to be treated. In the present invention, the patient is a human. In certain embodiments, the patient is an adult. In certain embodiments, the patient is an adult at least 50 years of age. In certain embodiments, the patient is an adult at least 60 years of age. In certain embodiments, the patient is a child.

### Combinations

The description above describes multiple aspects and embodiments of the invention, including therapeutic applications, treatment methods, and pharmaceutical compositions. The patent application specifically contemplates all combinations and permutations of the aspects and embodiments.

### III. EXAMPLES

### Chemistry

The invention now being generally described, will be more readily understood by reference to the following examples, which are included merely for purposes of illustration of certain aspects and embodiments of the present invention, and are not intended to limit the invention.

### Example 1 - Synthesis of a Compound of Formula (I) in which each R² and R³ is Deuterium

A compound of formula (I) in which each R² and R³ is deuterium (compound I-24) may be synthesized as shown in the following scheme.

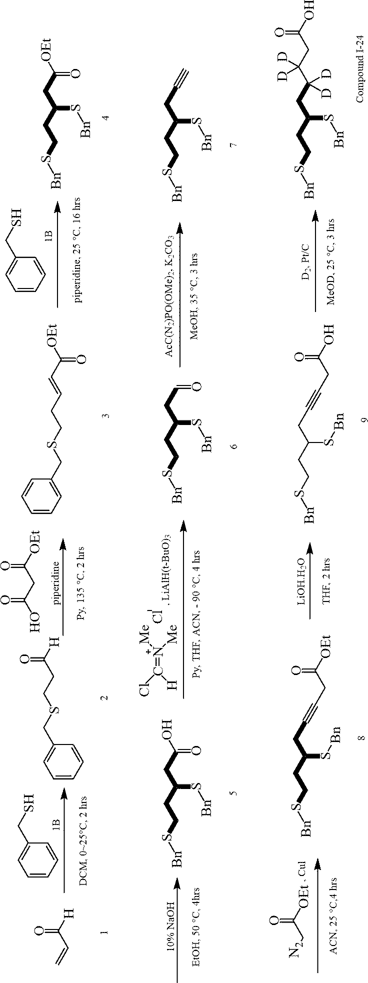

### Step 1: Synthesis of 3-(Benzylthio)Propanal (2)

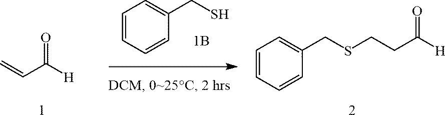

To a mixture of phenylmethanethiol 1B (63.6 g, 512 mmol, 60.0 mL, 1.00 eq) in DCM (750 mL) was added acrylaldehyde 1 (41.1 g, 696 mmol, 49.0 mL, 95.0% purity, 1.36 eq) in one portion at 0° C. under N₂, then heated to 25° C. and stirred for 2 hrs. TLC (petroleum ether/ethyl acetate = 5/1, R_{f} = 0.300) showed the starting material (acrylaldehyde 1) was consumed completely. The combined organic phase was concentrated in vacuo. Crude 3-(benzylthio)propanal 2 was obtained as colorless oil.

### Step 2: Synthesis of Ethyl 3,5-bis(Benzylthio)Pentanoate (4)

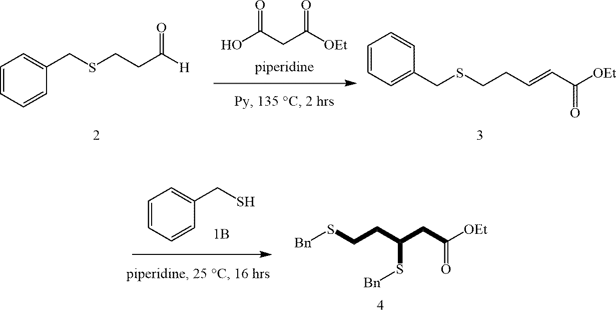

To a mixture of 3-(benzylthio)propanal 2 (65.0 g, 360 mmol, 1.00 eq) and 3-ethoxy-3-oxo-propanoic acid (47.6 g, 360 mmol, 1.00 eq) in Py (130 mL) was added piperidine (2.76 g, 32.4 mmol, 3.20 mL, 8.99e-2 eq) in one portion at 25° C., then the mixture was heated to 135° C. and stirred for 2 hrs to provide ethyl (E)-5-benzylsulfanylpent-2-enoate 3. To ethyl (E)-5-benzylsulfanylpent-2-enoate 3 (140 g, 559 mmol, 1.00 eq) in piperidine (23.8 g, 279 mmol, 27.6 mL, 0.500 eq) was added phenylmethanethiol 1B (69.4 g, 559 mmol, 65.5 mL, 1.00 eq) in one portion at 25° C. The resulting mixture was stirred at 25° C. for 3 hrs. LCMS (ET27289-78-P1A2, product: RT = 2.51 min; start material: RT = 2.07 min) showed the starting material (ethyl (E)-5-benzylsulfanylpent-2-enoate 3) was consumed completely. The residue was poured into ice-water (300 mL), and acidified to pH 2 with 6N HCl. The aqueous phase was extractedwith ethyl acetate (400 mL×3). The combined organic phase was washed with brine (200 mL), dried with anhydrous Na₂SO₄, and concentrated in vacuum. Ethyl 3,5-bis(benzylthio)pentanoate 4 (122 g, 325 mmol, 58.2% yield, crude) was obtained as yellow oil. LCMS: 374.1(M+1).

### Step 3: Synthesis of Ethyl 3,5-bis(benzylthio)pentanoate (5)

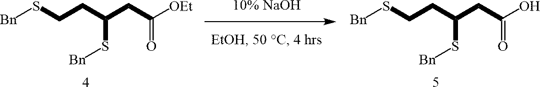

To a solution of ethyl 3,5-bis(benzylthio)pentanoate 4 (60.0 g, 160 mmol, 1.00 eq) in EtOH (240 mL) was added 10% NaOH (600 mL) in one portion at 25° C., heated to 50° C. and stirred for 4 hrs. LCMS (ET27289-81-P1A1, product: RT = 2.20 min; start material: RT = 2.51 min) showed the starting material was consumed completely. The reaction solution was concentrated under reduced pressure to remove EtOH. The residue was poured into ice-water (w/w = 1/1) (200 mL). The aqueous phase was extracted with MTBE (400 mL×3), the aqueous phase was acidified to pH 4 with 6N HCl. The aqueous phase was extracted with ethyl acetate (400 mL×3), the combined organic phase was washed with brine (300 mL×2), dried with anhydrous Na₂SO₄, concentrated in vacuum. The residue was purified by silica gel chromatography (column height: 250 mm, diameter: 100 mm, 100-200 mesh silica gel, petroleum ether/ethyl acetate = 30/1, 5/1). 3,5-bis(benzylthio)pentanoic acid 5 (24.0 g, 69.3 mmol, 43.2% yield) was obtained as yellow oil. LCMS:347.2(M+1). ¹H NMR (400 MHz, CDCl₃ 8 ppm 7.17-7.24 (m, 10H), 3.65 (s, 2H), 3.57 (s, 2H), 2.98-3.03 (m, 1H), 2.38-2.51 (m, 4H), 1.65-1.69 (m, 2H).

### Step 4: Synthesis of Ethyl 3,5-bis(Benzylthio)Pentanoate (6)

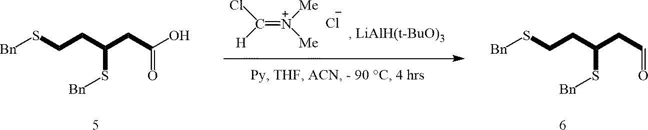

A mixture of chloromethylene(dimethyl)ammonium chloride (19.9 g, 155 mmol, 2.00 eq) in dry THF (290 mL) and dry CH₃CN (160 mL) was prepared at 25° C. under N₂ (mixture 1). A mixture of 3,5-bis(benzylthio)pentanoic acid 5 and Py (13.5 g, 170 mmol, 13.8 mL, 2.19 eq) in THF (290 mL) added dropwise to mixture 1 at -30° C. for 30 min under N₂. The resulting mixture was stirred at -30° C. for 1 hr, then heated to -20° C. and stirred for 30 min, then the mixture was cooled to -90° C., and LiA1H(t-BuO)₃ (1 M, 117 mL, 1.50 eq) in THF(100 mL) was added over 30 min. The resulting mixture was stirred at -90° C. for 30 min. TLC (petroleum ether/ethyl acetate = 5/1, R_{f} = 0.7) showed the reaction was complete. Then 2N HCl (108 mL) was added, and the cooling bath was removed. The aqueous phase was extracted with ethyl acetate (400 mL×3). The combined organic phase was washed with brine (300 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=25/1, 5/1). 3,5-bis(benzylthio)pentanal 6 (10.0 g, crude) was obtained as yellow oil. ¹H NMR (400 MHz, CDCl₃) 8 ppm 9.57 (t, J = 1.6 Hz, 1H), 7.33-7.28 (m, 10H), 3.73-3.70 (m, 2H), 3.68 (s, 2H), 3.10-3.15 (m, 1H), 2.56-2.52 (m, 2H), 2.49-2.55 (m, 1H), 1.77 (q, Ji = 7.2 Hz, J₂= 14.4 Hz, 2H)

### Step 5: Synthesis of Ethyl 3,5-bis(Benzylthio)Pentanoate (7)

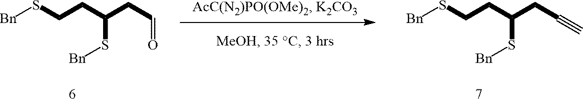

3,5-Bis(benzylthio)pentanal 6 (5.00 g, 15.1 mmol) and K₂CO₃ (8.36 g, 60.51 mmol) were added to dry MeOH (30.0 mL), and stirred for 0.5 hr at 0° C. under N₂. Then, AcC(N₂)PO(OMe)₂ (7.27 g, 37.8 mmol) was added at 0° C. under N₂, then the resulting mixture was stirred at 35° C. for 3 hrs under N₂. LCMS showed the reaction was completed. The mixture was poured into 30 mL DCM, filtered, and the filter cake was washed with DCM. The solution was evaporated on a water bath under reduced pressure using a rotary evaporator. The residue was purified by silica gel chromatography (column height: 250 mm, diameter: 100 mm, 100-200 mesh silica gel, Petroleum ether/Ethyl acetate=30/1, 10/1). The residue was purified by prep-HPLC (TFA condition, column: Phenomenex luna C18 250*80 mm*10 µm;mobile phase: [water(0.1%TFA)-ACN];B%: 58%-88%,20 min). Hex-5-yne-1,3-diylbis(benzylsulfane) 7 (2.20 g, 6.74 mmol, 22.3% yield) was obtained as yellow oil. LCMS: 327.3(M+1). ¹H NMR (400 MHz, CDCl₃ 8 ppm 6.92-7.44 (m, 10H), 3.71 (s, 2H), 3.67 (s, 2H), 2.70-2.74 (m, 2H), 2.33-2.44 (m, 4H), 1.96 (s, 1H), 1.95-1.96 (m, 1H), 1.68-1.70 (m, 1H)

### Step 6: Synthesis of Ethyl 3,5-bis(benzylthio)pentanoate (8)

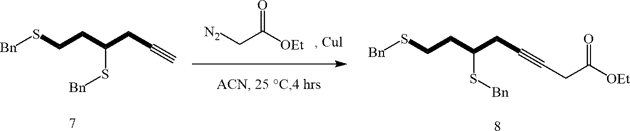

To a mixture of hex-5-yne-1,3-diylbis(benzylsulfane) 7 (2.00 g, 6.13 mmol) and CuI (583 mg, 3.06 mmol) in CH₃CN (20.0 mL) was added ethyl 2-diazoacetate (769 mg, 6.74 mmol, 705 µL,) in one portion at 25° C. The mixture was stirred at 25° C. for 4 hrs. LCMS showed the reaction was completed. The mixture was concentrated in vacuum, then isopropyl ether (25 mL) was added and the resulting mixture was filtered through silica, and the combined organic layers concentrated in vacuum. The residue was purified by silica gel chromatography (column height: 250 mm, diameter: 100 mm, 100-200 mesh silica gel, petroleum ether/ethyl acetate = 30/1, 10/1). Ethyl 6,8-bis(benzylthio)oct-3-ynoate 8 (250 mg, 606 µmol, 9.89% yield) was obtained as yellow oil. LCMS: 413.2(M+1). ¹H NMR (400 MHz, CDCl₃) 8 ppm 7.16-7.33 (m, 10H), 4.10 (q, Jᵢ = 6.8 Hz, J₂ = 14 Hz, 2H), 3.67 (s, 2H), 3.58 (s, 2H), 3.17 (t, J=’2.4 Hz, 2H), 2.69-2.71 (m, 1H), 2.35-2.46 (m, 4H), 1.90-1.91 (m, 1H), 1.71-1.67 (m, 1H), 1.20 (t, J=4.8 Hz, 3H).

### Step 7: Synthesis of 6,8-bis(benzylthio)oct-3-ynoic Acid (9)

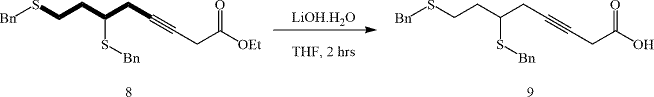

To a mixture of ethyl 6,8-bis(benzylthio)oct-3-ynoate 8 (50.0 mg, 121 pmol) in THF (2.0 mL) was added LiOH.H₂O (30.5 mg, 727 µmol) in one portion at 25° C. and the resulting mixture was stirred for 2 hrs. LCMS showed the reaction was completed. The residue was poured into ice-water (w/w = 1/1) (2.0 mL). The aqueous phase acidified to pH 4 with 1N HCl. The aqueous phase was extracted with DCM (5 mL×3). The combined organic phase was washed with brine (5.0 mL), dried with anhydrous Na₂SO₄, and concentrated in vacuum. 6,8-bis (benzylthio)oct-3-ynoic acid 9 (46.0 mg, 98.7% yield, crude) was obtained as colorless oil. LCMS: 383.2(M-1).

### Step 8: Synthesis of 6,8-bis(Benzylsulfanyl)-3,3,4,4-Tetradeuterio-Octanoic Acid (I-24)

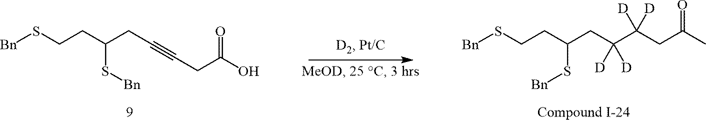

To a solution of 6,8-bis(benzylthio)oct-3-ynoic acid 9 (20.0 mg, 26.0 µmol) in MeOD (6.00 mL) was added Pd/C (60.0 mg, 10% purity) under D₂. The mixture was stirred under D₂ (15 psi) at 25° C. for 3 hours. LCMS showed the reaction was completed. The residue was purified by prep-HPLC (neutral condition, column: Waters Xbridge BEH C18 100*25 mm*5 µm; mobile phase: [water (10 mM NH₄HCO₃)-ACN]; B%: 25%-50%, 10 min). 6,8-bis(benzylsulfanyl)-3,3,4,4-tetradeuterio-octanoic acid Compound I-24 (3.00 mg, 14.7% yield) was obtained as pale colorless liquid. LCMS: 391.1(M-1); ¹H NMR (400 MHz, CDCl₃ 8 ppm 7.30-7.25(m, 10H), 3.64(d, J = 10.0 Hz, 4H), 2.55(t, J = 6.8 Hz, 1H), 2.48 (t, J = 7.2 Hz, 2H), 2.26(s, 2H), 1.71(q, J₁ = 7.6 Hz, J₂ = 14.8 Hz, 2H), 1.43(d, J = 6.80 Hz, 2H).

### Example 2 - Synthesis of a Compound of Formula (I) in which each R² is Deuterium

A compound of formula (I) in which each R² is deuterium (compound I-10) may be synthesized as shown in the following scheme.

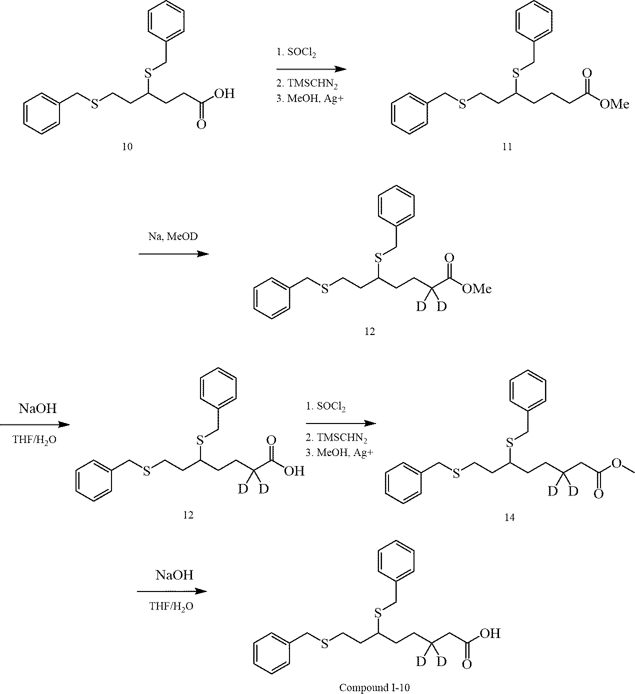

### Step 1: Synthesis of Methyl 5,7-bis(benzylsulfanyl)heptanoate (11)

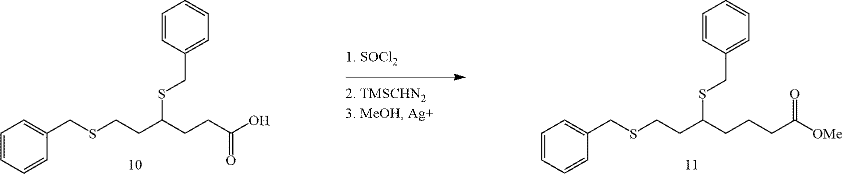

Sulfinyl chloride (485.6 µL, 5.55 mmol) was added dropwise to a solution of 4,6-bis(benzylsulfanyl)hexanoic acid 10 (1 g, 2.77 mmol) and DMF (21 µL, 277.4 µmol) in DCM (15 mL) at 0° C. Then the mixture was stirred at 15° C. for 0.5 h and concentrated under vacuum to give an oily residue, the residue was dissolved in THF (15 mL), and TMSCHN₂ (2 M, 13.9 mL) was added to the mixture at 15° C. Then the mixture was stirred at 15° C. for 3 h and then the mixture was concentrated to remove THF to give a residue. The residue was dissolved in MeOH (15 mL). A mixture of TEA (1.40 g, 13.9 mmol, 1.9 mL) and benzoyloxy silver (158.8 mg, 693 µmol) was added to the mixture at 15° C. and stirred at 15° C. for 1 h. The mixture was filtered and the filtrate was concentrated to give a residue. The residue was purified by column (SiO₂, Petroleum ether/Ethyl acetate = 100/1 to 10/1) to give methyl 5,7-bis (benzylsulfanyl) heptanoate 11 (600 mg, 55.7% yield) as a yellow oil. LCMS: 389.2(M+1).

### Step 2: Synthesis of Methyl 5,7-bis(Benzylsulfanyl)-2,2-Dideuterio-Heptanoate (12)

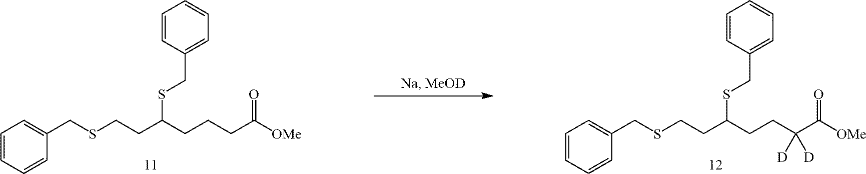

Na (17.8 mg, 0.772 mmol) was added to MeOH-D₄ (5 mL) at 15° C. under N₂, and the resulting mixture was stirred for 10 min. Then, methyl 5,7-bis (benzylsulfanyl) heptanoate 11 (300 mg, 0.772 mmol) was added to the mixture. The mixture was stirred at 60° C. for 24 h. ¹HNMR showed-95% of desired compound was detected. The reaction was quenched with D₂O (0.5 mL) at 15° C. and concentrated to give methyl 5,7-bis (benzylsulfanyl)-2,2-dideuterio-heptanoate 12 (302 mg, crude) as a yellow oil. LCMS: 391.2(M+1).

### Step 3: Synthesis of 5,7-bis(Benzylsulfanyl)-2,2-Dideuterio-Heptanoic Acid (13)

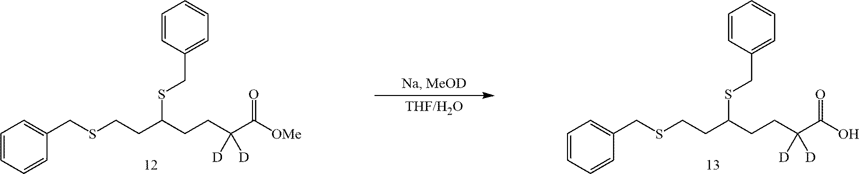

NaOH (61.4 mg, 1.54 mmol, 2 eq) was added to a solution of methyl 5,7-bis(benzylsulfanyl)-2,2-dideuterio-heptanoate 12 (300 mg, 768 µmol, 1 eq) in THF (3 mL) and H₂O (1 mL) at 15° C., then the mixture was stirred at 60° C. for 1 h. The mixture was acidified to pH=3, extracted with EtOAc (10 ml), washed with water (50 ml) and brine (10 ml), dried over Na₂SO₄, then concentrated to give 5,7-bis(benzylsulfanyl)-2,2-dideuterio-heptanoic acid 13 (0.238 g) as a yellow oil. LCMS: 375.2 (M-1).

### Step 4: Synthesis of Methyl 6,8-bis(Benzylsulfanyl)-3,3-Dideuterio-Octanoate (14)

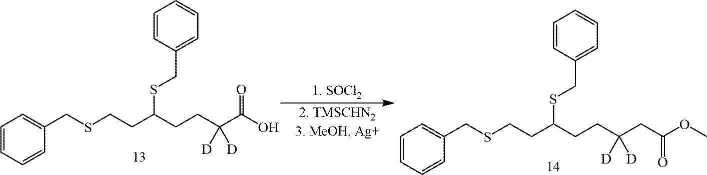

Sulfinyl chloride (160.4 mg, 1.26 mmol) was added dropwise to a solution of 5,7-bis(benzylsulfanyl)-2,2-dideuterio-heptanoic acid 13 (238 mg, 632 µmol) and DMF (4.6 mg, 63.20 µmol) in DCM (3 mL) at 0° C. Then the mixture was stirred at 15° C. for 0.5 h. Then concentrated the mixture to give a residue, the residue was dissolved in THF (3 mL) and TMSCHN₂ (2 M, 3.16 mL) was added to this mixture at 15° C. The mixture was stirred at 15° C. for 3 h and the mixture was concentrated to remove THF to give a residue. The residue was dissolved in MeOH (3 mL). Then a mixture of TEA (319.8 mg, 3.16 mmol) and benzoyloxy silver (36.2 mg, 158 µmol) was added to the mixture at 15° C. and stirred at 15° C. for 1 h. The mixture was filtered and the filtrate was concentrated to give a residue. The residue was purified by prep-TLC (Petroleum ether/Ethyl acetate=5/1) to give methyl 6,8-bis(benzylsulfanyl)-3,3-dideuterio-octanoate 14 (60 mg, 22.7% yield) as a yellow oil. LCMS: 405.2 (M+1).

### Step 5: Synthesis of 6,8-bis(Benzylsulfanyl)-3,3-Dideuterio-Octanoic Acid (1-10)

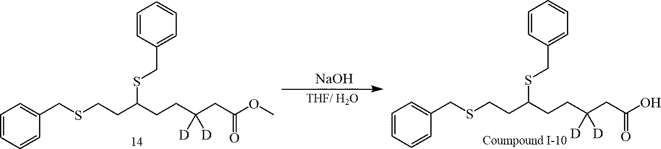

NaOH (11.9 mg, 0.30 mmol) was added to a solution of methyl 6,8-bis (benzylsulfanyl)-3,3-dideuterio-octanoate 14 (60 mg, 0.148 mmol) in THF (1 mL) and H₂O (0.3 mL) at 15° C. Then the mixture was heated to 60° C. and stirred for 1 h. The mixture was acidified to pH=3 by adding 1M HCl, then the mixture was extracted with EtOAc (10 ml×2). The organic layer was concentrated to give a residue. The residue was purified by prep-TLC (SiO₂, Petroleum ether/Ethyl acetate=5/1) to give 6,8-bis(benzylsulfanyl)-3,3-dideuterio-octanoic acid 1-10 (30 mg, 48.0%) as a colorless oil. LCMS:389.2(M-1). ¹H NMR (400 MHz, METHANOL-d₄) 8 ppm 7.14 - 7.36 (m, 10 H), 3.67 (s, 2 H), 3.65(s, 2 H), 2.47 - 2.58 (m, 3 H), 2.20 (s, 2 H), 1.67 -1.71 (m, 2 H), 1.26 - 1.44 (m, 4 H).

### Example 3 - Synthesis of Compound 1-77

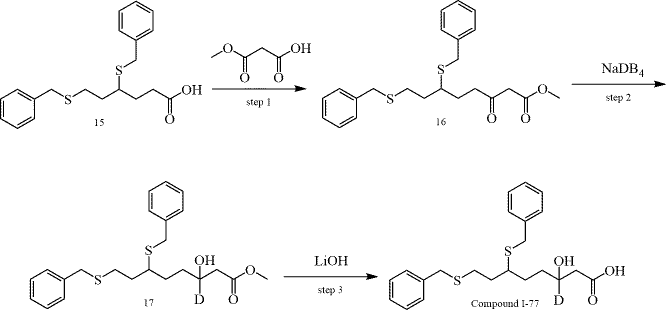

### Step 1: Synthesis of Compound 15

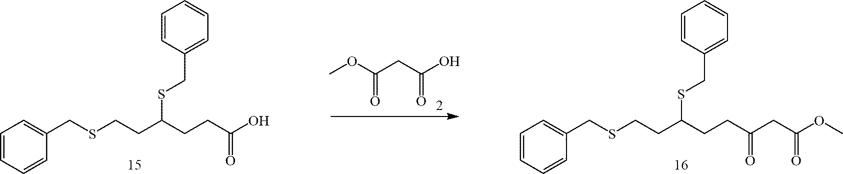

Carbonyldiimidazole (0.5 g, 3 mmol) was added to a solution of compound 15 (0.9 g, 2.5 mmol) in THF (10 ml) and the mixture was stirred at room temperature for 1 hour. To the reaction solution was added magnesium chloride (0.25 g, 2.5 mmol) and potassium 3-methoxy-3-oxopropanoate (0.4 g, 2.5 mmol), and then the resulting mixture was stirred at 60° C. for 1 hour. The mixture was filtered to remove insoluble substances and the filtrate was concentrated under reduced pressure. The residue was diluted with water, acidified and then extracted with ethyl acetate (10 ml) twice. The extracts were washed with an aqueous saturated sodium chloride solution, dried over sodium sulfate, and then concentrated under vacuum. The residue was purified by silica gel column chromatography to obtain 0.35 g of compound 16 as a colorless oil (34% yield). LCMS: 417.2 (M+1).

### Step 2: Synthesis of Compound 17

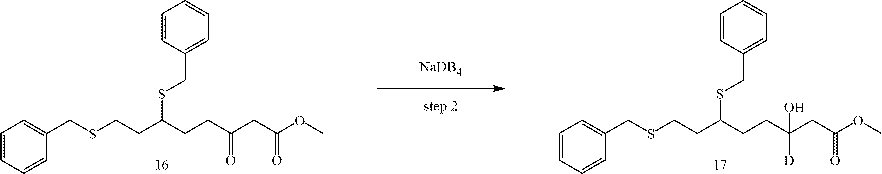

To a solution of Compound 16 (0.21 g, 0.5 mmol) in DCM (4 mL) was added AcOD (0.3 g, 5 mmol) at 0° C. After stirring the mixture for 10 min, NaBD4 (0.04 g, 1 mmol) was added in portions. The mixture was stirred for an hour at 0° C. The resulting solution was washed with saturated aqueous NaHCO₃ (2×10 mL) and saturated aqueous NaCl (10 mL), dried over anhydrous Na₂SO₄ and concentrated under vacuum. The residue was purified by silica gel column chromatography to give 65 mg of compound 17 as a colorless oil (31% yield). LCMS: 420.2 (M+1).

### Step 3: Synthesis of Compound I-77

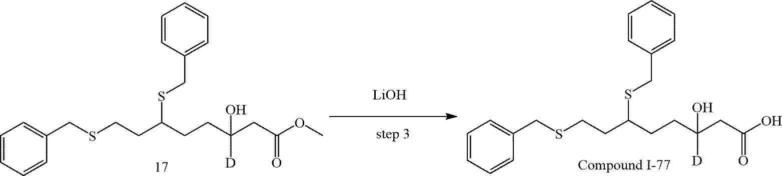

To a solution of compound 17 (0.08 g, 0.02 mmol) in MeOH (4 mL) was added LiOH·H₂O (16 mg, 0.04 mmol), and the resulting mixture was stirred overnight at RT. The resulting solution was acidified with aq. HCl and concentrated. The residue was dissolved in ethyl acetate and washed with water, then dried over anhydrous Na₂SO₄. The organic layer was filtered and concentrated, followed by purification by silica gel chromatography (30% EtOAc/hexanes) to yield the title compound I-77 as a colorless oil (35 mg, 43%). LCMS: 404.2 (M-1).

### Example 4: Synthesis of Compound I-78

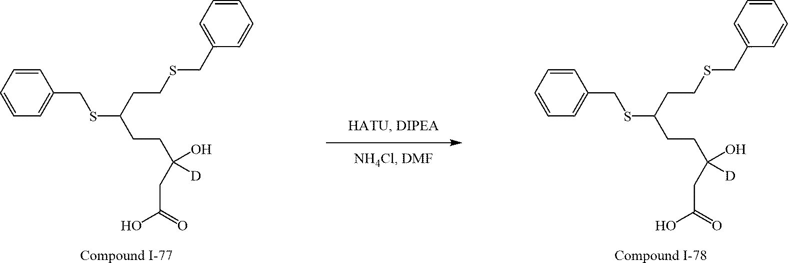

Compound I-77(40 mg, 0.1 mmol) in DMF (1 mL) was treated with HATU (38 mg, 0.1 mmol) and DIEA (0.1 mL, 0.2 mmol). After stirring for 30 min at RT, NH₄Cl (10 mg, 0.2 mmol) was added directly into the reaction mixture. The reaction was stirred overnight at RT. The remaining solution was partitioned between brine (2×25 mL) and DCM (100 mL). The combined DCM layers were stirred with 1N HCl (2×25 mL) for 15 min. The mixture was then re-extracted into DCM (1×50 mL). The DCM layer was washed with brine (2×50 mL) and dried over sodium sulfate. The organic layer was filtered and concentrated, followed by purification by silica gel chromatography (30% EtOAc/hexanes) to yield the title compound I-78 as a colorless oil (36 mg, 90%). LCMS: 405.2 (M+1).

### Example 5: Synthesis of Compound I-79

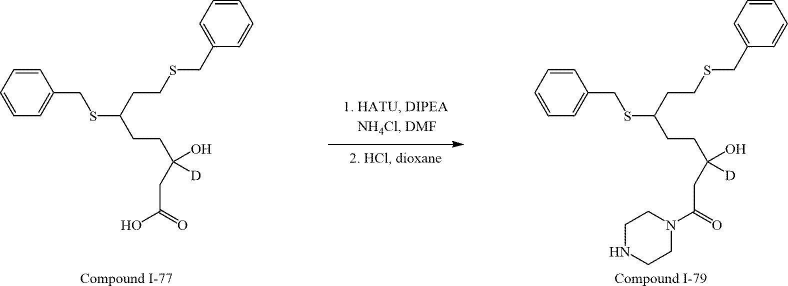

Compound I-77(40 mg, 0.1 mmol) in DMF (1 mL) was treated with HATU (38 mg, 0.1 mmol) and DIEA (0.1 mL, 0.2 mmol). After stirring for 30 min at RT, Boc-piperazine (16 mg, 0.2 mmol) was added directly into the reaction mixture. The reaction was stirred overnight at RT. The remaining solution was partitioned between brine (2×25 mL) and DCM (100 mL). The combined DCM layers were stirred with 1N HCl (2×25 mL) for 15 min. The mixture was then re-extracted into DCM (1×50 mL). The DCM layer was washed with brine (2×50 mL) and dried over sodium sulfate. The organic layer was filtered and concentrated, followed by purification by silica gel chromatography (30% EtOAc/hexanes) to yield the intermediate as acolorless oil. This intermediate was dissolved in dioxane (1 mL) was treated with 4N HCl (4 ml). After stirring overnight at RT, the remaining solution was evaporated to give the crude product. The crude product was washed with Hex/EA to remove impurities, to provide an insoluble solid that is the pure final product I-79(20 mg, 42%). LCMS: 474.2 (M+1).

### Biology

Biological activity was assessed in vitro in the following pancreatic cancer and colorectal cancer cell lines.
Pancreatic cancer cell lines
   Panc-1: non-metastatic; mutations: KRAS, TP53
   AsPC-1: metastatic; mutations: KRAS, TP53, CDKN2A, MAP2K4
Colorectal cancer cell lines
   LoVo: metastatic; mutations: APC, KRAS, FBXW7, MSH1
   SW620: metastatic; mutations: KRAS, TP53

Tumor cells were maintained at 37° C. in a humidified 5% CO₂ atmosphere in T75 tissue culture flask containing 10 mL of RPMI 1640 (PANC-1), DMEM (AsPC-1), F12K (LoVo) or L15 (SW620), containing 2 mM L-glutamine, 10% fetal bovine serum (FBS) and 1% penicillin and streptomycin. The tumor cells were split at a ratio of 1:5 every 4-5 days by trypsinization and suspended in fresh medium in a new flask. Cells were harvested for experiments at 80-95% confluency.

PANC-1 cells in RPMI medium with 10% FBS (12,000 cells/well in 96 well plate) were incubated with increasing concentrations (12.5, 25, 50, 100, 150, and 200 µM), of test compound at 37° C. under 5% CO₂ for 48 hours. Cells were undergone for another 24 hr recovery, in fresh RPMI media without serum. Cell viability was assessed using the Promega Cell Titer-Glo assay kit, following their protocol. Cell viability was measured based on the amount of ATP produced by the total number of cells present in each well using FLUOSTAR OPTIMA plate reader in the Luminescence mode. Production of ATP in Luminescence unit is directly proportional to the number of live cells. % live cells were calculated and SEM was also calculated as average of 3 replica wells. Results are presented in Table 2.

Test compounds were screened for activity in AsPC-1 cells and the colon cancer cells using similar procedures but replacing RPMI with the respective media indicated above for the initial 48 hour incubation (i.e., DMEM (AsPC-1), F12K ( LoVo ), or L15 (SW620)), and RPMI was always used for the 24 hour recovery. Cells seeded for AsPc-1 (25,000 cells/well), LoVo (25,000 cells/well) and SW620 (25,000 cells/well) to achieve ~ 50% confluency after 24 hrs of seeding. Results are presented in Table 2.

TABLE 2

IC₅₀/IC₉₀ (µM) of Test Compounds in Various Cells/Replete Media

| Compound | Panc-1 | LoVo | AsPC-1 | SW620 |
|---|---|---|---|---|
| I-10 | >300 | >300 | | |
| I-24 | >300 | >300 | | |
| I-77 | >300 | >300 | 314 | >300 |
| I-78 | e/73 | 26/39 | 28/65 | 28/37 |
| I-79 | 44/282 | 181/254 | | |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound of formula (I): 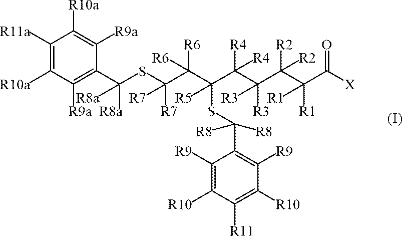
or a salt thereof, wherein:
X is —OH or -NR20R21;
each R1, R3, R4, R5, R6, R7, R8, R8a, R9, R9a, R10, R10a, R11, and R11a is independently chosen from hydrogen and deuterium;
each R2 is independently chosen from hydrogen, deuterium, —OH, and -O(C₁-C₆ alkyl);
R20 and R21 are independently chosen from H and C₁-C₄ alkyl; or R20 and R21 are taken together with the nitrogen atom to which they are attached to form a 5-10 membered heterocycloalkyl, which is optionally substituted by one or more R30 groups;
each R30 is independently chosen from halogen, C₁-C₆ alkyl, —OH, -O(C₁-C₆ alkyl), —NH₂, -NH(C₁-C₆ alkyl), and -N(C₁-C₆ alkyl)₂;
when any combination of R1, R2, R3, R4, R5, R6, R7, R8, R8a, R9, R9a, R10, R10a, R11, and R11a is deuterium, each deuterated position has greater than 50 percent incorporation of deuterium; and
when each R1, R3, R4, R5, R6, R7, R8, R8a, R9, R9a, R10, R10a, R11, and R11a is hydrogen, then at least one R2 is deuterium, —OH, or -O(C₁-C₆ alkyl).

2. A compound of formula (I) of claim 1 or a salt thereof, wherein X is —OH.

3. A compound of formula (I) of claim 1 or a salt thereof, wherein X is -NR20R21.

4. A compound of formula (I) of claim 3 or a salt thereof, wherein R20 and R21 are taken together with the nitrogen atom to which they are attached to form a 5 or 6 membered heterocycloalkyl optionally substituted by one or more R30 groups.

5. A compound of formula (I) of claim 4 or a salt thereof, wherein R20 and R21 are taken together with the nitrogen atom to which they are attached to form a 6 membered heterocycloalkyl optionally substituted by one or more R30 groups.

6. A compound of formula (I) of any of claims 1-5 or a salt thereof, wherein each R2 is independently chosen from hydrogen, deuterium, and —OH.

7. A compound of formula (I) of any of claims 1-6 or a salt thereof, wherein each R2 is deuterium; each R1, R3, R4, R6, R7, R8, R8a, R9, R9a, R10, and R10a is hydrogen; and R5, R11, and R11a are each hydrogen.

8. A compound of formula (I) of any of claims 1-6 or a salt thereof, wherein each R2 and R3 is deuterium; each R1, R4, R6, R7, R8, R8a, R9, R9a, R10, and R10a is hydrogen; and R5, R11, and R11a are hydrogen.

9. A compound of formula (I) of any of claims 1-6 or a salt thereof, wherein one R2 and one R3 is deuterium, and one R2 and one R3 is hydrogen; each R1, R4, R6, R7, R8, R8a, R9, R9a, R10, and R10a is hydrogen; and R5, R11, and R11a are hydrogen.

10. A compound of formula (I) of any of claims 1-6 or a salt thereof, wherein each R2, R3, R4, R6, and R7 is deuterium; each R1, R8, R8a, R9, R9a, R10, and R10a is hydrogen; R5 is deuterium; and R11 and R11a are hydrogen.

11. A compound of formula (I) of any of claims 1-6 or a salt thereof, wherein each R2, R3, and R4, is deuterium; each R1, R6, R7, R8, R8a, R9, R9a, R10, and R10a is hydrogen; and R5, R11, and R11a are hydrogen.

12. A compound of formula (I) of any of claims 1-6 or a salt thereof, wherein each R1, R2, R3, R4, R5, R6, R7, R8, R8a, R9, R9a, R10, R10a, R11, and R11a is deuterium.

13. A compound of formula (I) of any of claims 1-6 or a salt thereof, wherein each R2, R3, R4, R5, R6, R7, R8, R8a, R9, R9a, R10, R10a, R11, and R11a is deuterium, and each R1 is hydrogen.

14. A compound of formula (I) of any of claims 1-6 or a salt thereof, wherein each R1, R2, R3, R4, R5, R6, and R7 is deuterium; and each R8, R8a, R9, R9a, R10, R10a, R11, and R11a is hydrogen.

15. A compound of formula (I) of any of claims 1-6 or a salt thereof, wherein each R2, R3, R4, R5, R6, and R7 is deuterium; and each R1, R8, R8a, R9, R9a, R10, R10a, R11, and R11a is hydrogen.

16. A compound of formula (I) of any of claims 1-6 or a salt thereof, wherein each R1, R2, R3, R8, R8a, R9, R9a, R10, R10a, R11, and R11a is deuterium; and each R4, R5, R6, and R7 is hydrogen.

17. A compound of formula (I) of any of claims 1-6 or a salt thereof, wherein each R2, R3, R8, R8a, R9, R9a, R10, R10a, R11, and R11a is deuterium; and each R1, R4, R5, R6, and R7 is hydrogen.

18. A compound of formula (I) of any of claims 1-6 or a salt thereof, wherein each R2, R8, R8a, R9, R9a, R10, R10a, R11, and R11a is deuterium; and each R1, R3, R4, R5, R6, and R7 is hydrogen.

19. A compound of formula (I) of any of claims 1-6 or a salt thereof, wherein each R2, R8, and R8a is deuterium; and each R1, R3, R4, R5, R6, R7, R9, R9a, R10, R10a, R11, and R11a is hydrogen.

20. A compound in Table 1 herein, or a salt thereof.

21. A compound of any of claims 1 to 20, wherein each deuterated position has at least 90 percent incorporation of deuterium.

22. A pharmaceutical composition comprising a compound of any of claims 1 to 21, and a pharmaceutically acceptable carrier.

23. A method of treating cancer in a human patient in need thereof, comprising the step of administering to the patient a therapeutically effective amount of a pharmaceutical composition of claim 22 to treat the cancer.

24. The method of claim 23, wherein the cancer is a lymphoma, leukemia, carcinoma, sarcoma, myeloma, or a cancer of the pancreas.
